(19) United States
(12) Patent Application Publication
(10) Pub. No.: US 2018/0299458 A1
(43) Pub. Date: Oct. 18, 2018

(54) PLASMONIC NANOPARTICLES AND LSPR-BASED ASSAYS

(71) Applicant: LamdaGen Corporation, Menlo Park, CA (US)

(72) Inventors: Daniele Gerion, Oakland, CA (US); Randolph Storer, Hillborough, CA (US)

(21) Appl. No.: 15/817,598

(22) Filed: Nov. 20, 2017

Related U.S. Application Data
(63) Continuation of application No. PCT/US16/33633, filed on May 20, 2016.
(60) Provisional application No. 62/165,119, filed on May 21, 2015.

(51) Int. Cl. G01N 33/58; B01J 13/02; G01N 33/68; G01N 33/74

(52) U.S. Cl. CPC G01N 33/587; B01J 13/02; B82Y 20/00; G01N 33/74; G01N 33/689

(57) ABSTRACT

Compositions, methods, devices, and systems are described for performing single-step, homogenous, localized surface plasmon resonance (LSPR)-based plasmonic assays having exceptional assay sensitivity and extremely low limits of detection (LODs). Ag/Au core/shell nanoparticles are described, which may be used with LSPR sensors to develop single-step, homogeneous, LSPR-based assays.

### CROSS-REFERENCE

This application is a Continuation Application of International Patent Application PCT/US2016/033633, filed May 20, 2016, which claims the benefit of U.S. Provisional Application No. 62/165,119, filed May 21, 2015, each of which is incorporated herein in its entirety by reference.

### BACKGROUND OF THE INVENTION

The accuracy, sensitivity, reproducibility, and ease-of-use of instruments designed for detection and quantitation of specific molecules (e.g., analytes or markers) and/or analysis of molecular interactions are of paramount concern in a variety of fields including biomedical research, clinical diagnostics, environmental testing, and industrial process monitoring. These concerns are driven by a variety of factors including the difficulty and cost associated with producing and/or isolating molecules of interest in biomedical research, for example, or the critical impact that a diagnostic test result may have on proper diagnosis and treatment of disease in the healthcare field. Often, molecules may be present in samples of interest only at very low concentrations and may require extremely sensitive assays for detection. While a variety of assay procedures and detection techniques exist, they are often insufficient to detect analytes that are present in samples in minute quantities. Therefore, a continuing need exists to improve the sensitivity, limit of detection, quantitation, and/or time-to-result required for assay devices and instruments, and especially for those intended for use in field testing or point-of-care diagnostics testing applications. Improvements in signal amplification and/or detection techniques will play an important role in achieving these objectives.

### SUMMARY OF THE INVENTION

Disclosed herein are nanoparticle compositions comprising: a) a silver (Ag) core; b) a gold (Au) shell partially or wholly encapsulating the silver core, wherein the thickness of the gold shell is substantially less than the diameter of the silver core; and c) a polymer layer partially or wholly encapsulating the Ag core and the Au shell. In some embodiments, the silver core has a shape that is consistent with a cubic close-packed crystal structure, i.e., roughly triangular or hexagonal in two dimensions. In some embodiments, the silver core has a long axis dimension ranging from 30 nm to 100 nm. In some embodiments, the silver core has a short axis dimension (thickness) ranging from 5 nm to 10 nm. In some embodiments, the gold shell has a thickness of between 1 and 20 atomic layers. In some embodiments, the polymer layer stabilizes the metal particle core. In some embodiments, the polymer layer is between 1 nm and 50 nm thick. In some embodiments, the polymer is selected from the group consisting of poly-vinyl-pyrrolidone (PVP), poly-vinyl-alcohol (PVA), polyacrylates, and combinations thereof. In some embodiments, the nanoparticles are immobilized on a surface. In some embodiments, the surface is an LSPR-active surface. In some embodiments, two or more nanoparticles form clusters or aggregates. In some embodiments, the nanoparticle has an average dimension ranging from 20 nm to 80 nm. In some embodiments, the nanoparticle has an average dimensions ranging from 40 nm to 60 nm. In some embodiments, the nanoparticle composition further comprises a biomolecule layer conjugated to the gold shell. In some embodiments, the biomolecule layer comprises molecules selected from the group consisting of proteins, peptides, antibodies, antibody fragments, oligonucleotides, and any combination thereof. In some embodiments, the biomolecule layer is conjugated to the thin gold shell using a bifunctional cross-linker comprising a mercapto group.

Also disclosed herein are methods for producing core-shell nanoparticles comprising: a) reducing silver ions in solution to metallic silver, thereby producing silver (Ag) core nanoparticles; b) rinsing the silver colloidal particles produced in step (a) to produce silver core nanoparticles having a stable plasmon resonance peak in the range of 400-680 nm; and c) growing an epitaxial gold (Au) shell on the silver core nanoparticles produced in step (b) in the presence of a polymer solution to thereby generate Ag/Au core-shell nanoparticles. In some embodiments, sodium borohydride is used as a reducing agent. In some embodiments, the reducing by sodium borohydride is performed in the presence of trisodium citrate and hydrogen peroxide. In some embodiments, step (b) is repeated two or more times to produce silver core nanoparticles having a stable plasmon resonance peak in the range of 450 to 480 nm. In some embodiments, the polymer is selected from the group consisting of poly-vinyl-pyrrolidone (PVP), poly-vinyl-alcohol (PVA), polyacrylates, and combinations thereof. In some embodiments, the polymer has a molecular weight in the range of 3,500 Da to 50,000 Da. In some embodiments, a ratio of a concentration of the polymer to a concentration of the silver core nanoparticles used in step (c) has a value in the range of 10³ to 10⁹. In some embodiments, the silver core nanoparticles have a triangular or hexagonal shape in two dimensions consistent with a cubic close-packed crystal structure, and a long axis dimension ranging from 30 nm to 100 nm. In some embodiments, the silver core nanoparticles have a short axis dimension (thickness) ranging from 5 nm to 10 nm. In some embodiments, the gold shell has a thickness of between 1 and 20 atomic layers. In some embodiments, the method further comprises conjugating a layer of biomolecules to the gold shell. In some embodiments, the biomolecules are selected from the group consisting of proteins, peptides, antibodies, antibody fragments, oligonucleotides, and any combination thereof. In some embodiments, the biomolecules are conjugated to the gold shell using a bifunctional cross-linker comprising a mercapto group.

Disclosed herein are methods for producing core-shell nanoparticles comprising: a) reducing silver ions in solution to metallic silver, thereby producing silver (Ag) core nanoparticles; and b) growing an epitaxial gold (Au) shell on the silver core nanoparticles produced in step (a) in the presence of a polymer to stabilize the silver core nanoparticles, thereby generating Ag/Au core-shell nanoparticles; wherein a ratio of a concentration of the polymer to a concentration of the silver core nanoparticles used in step (b) has a value in the range of 10³ to 10⁹. In some embodiments, the method further comprises rinsing the silver core nanoparticles produced in step (a) two or more times to produce silver core nanoparticles having a stable plasmon resonance peak in a range of 450 to 480 nm. In some embodiments, sodium borohydride is used as a reducing agent. In some embodiments, the reducing by sodium borohydride is performed in the presence of trisodium citrate and hydrogen peroxide. In some embodiments, the method further comprises conjugating a layer of biomolecules to the gold shell. In some embodiments, the biomolecules are selected from the group consisting of proteins, peptides, antibodies, antibody fragments, oligonucleotides, and any combination thereof. In some embodiments, the biomolecules are conjugated to the gold shell using a bifunctional cross-linker comprising a mercapto group. In some embodiments, the polymer is selected from the group consisting of poly-vinyl-pyrrolidone (PVP), poly-vinyl-alcohol (PVA), polyacrylates, and combinations thereof. In some embodiments, the polymer has a molecular weight in the range of 3,500 Da to 50,000 Da. In some embodiments, the gold shell has a thickness of between 1 and 20 atomic layers.

Disclosed herein are methods for detection of analytes in a sample comprising: a) mixing a sample containing one or more analytes of interest with one or more secondary binding components conjugated to metal nanoparticles, wherein the one or more secondary binding components are capable of specifically binding to the one or more analytes of interest; b) contacting an LSPR surface with the mixture of step (a), wherein the LSPR surface has been functionalized with one or more primary binding components that are capable of specifically binding to the one or more analytes of interest; and c) detecting a change in a physical property of light transmitted by or reflected from the LSPR surface; wherein the plasmon resonance properties of the metal nanoparticles and those of the LSPR surface are adjusted to substantially match, thereby providing improved detection sensitivity. In some embodiments, the metal nanoparticles are selected from the group consisting of Au nanoparticles, Ag/Au core/shell nanoparticles, or hybrid magnetic/plasmonic nanoparticles. In some embodiments, the one or more analytes are selected from the group consisting of a peptide, a protein, an oligonucleotide, a DNA molecule, an RNA molecule, a virus, a bacterium, a cell, a pathogen, a lipid molecule, a carbohydrate molecule, a small organic molecule, a drug molecule, an ion, creatinine, lactate, C-reactive protein (CRP), alpha-fetoprotein (AFP), cardiac troponin I (cTnI), cardiac troponin T (cTNT), cardiac phosphocreatine kinases M and B (CK-MB), brain natriuretic peptide (BNP), cortisol, S100BB, tau protein, thyroid-stimulating hormone (TSH), circulating tumor cells (CTC's), and any combination thereof. In some embodiments, the sample is selected from the group consisting of air, water, soil, a gas, an industrial process stream, feces, biological tissue, cells, blood, plasma, serum, sweat, tears, urine, cerebral spinal fluid, saliva, and any combination thereof. In some embodiments, the primary and secondary binding components are selected from the group consisting of antibodies, antibody fragments, aptamers, molecularly imprinted polymers, biotin, streptavidin, his-tags, chelated metal ions such as Ni-NTA, receptors, enzymes, peptides, proteins, oligonucleotide probes, and any combination thereof. In some embodiments, the plasmon resonance properties of the Ag/Au core/shell nanoparticles are adjusted by a method selected from the group consisting of changing the number of rinse steps used to rinse Ag core nanoparticles used to fabricate the Ag/Au core/shell nanoparticles, changing the size of Ag core nanoparticles used to fabricate the Ag/Au core/shell nanoparticles, changing the shape of Ag core nanoparticles used to fabricate the Ag/Au core/shell nanoparticles, changing the thickness of an Au shell used to fabricate the Ag/Au core/shell nanoparticles, and any combination thereof. In some embodiments, the LSPR surface is a nanostructured LSPR surface. In some embodiments, the plasmon resonance properties of the nanostructured LSPR surface are adjusted by a method selected from the group consisting of changing the choice of materials used to fabricate the LSPR surface, changing the dimensions of the layers of material used to fabricate the LSPR surface, changing the number of layers of material used to fabricate the LSPR surface, changing the order of the layers used to fabricate the LSPR surface, and any combination thereof. In some embodiments, the change in a physical property of light transmitted by or reflected from the LSPR surface is a color change that is detected visually to provide a qualitative assay result. In some embodiments, the physical property of light transmitted by or reflected from the LSPR surface is detected using one or more detectors to provide a qualitative or quantitative assay result. In some embodiments, the change in a physical property of light transmitted by or reflected from the LSPR surface is a shift in the plasmon absorption peak. In some embodiments, the physical property of light transmitted by or reflected from the LSPR surface is selected from the group consisting of intensity, spectrum, polarization, angle of reflection, and changes in RGB or greyscale values. In some embodiments, a limit of detection (LOD) for the method is better than 1 ug/mL. In some embodiments, a limit of detection (LOD) for the method is better than 1 ng/mL. In some embodiments, a limit of detection (LOD) for the method is better than 100 pg/mL. In some embodiments, a limit of detection (LOD) for the method is better than 10 pg/mL. In some embodiments, a limit of detection (LOD) for the method is better than 1 pg/mL. In some embodiments, a limit of detection (LOD) for the method is better than 0.1 pg/mL. In some embodiments, the method further comprises determination of a concentration of the one or more analytes. In some embodiments, the method is performed as a single-step assay that provides a result in 30 minutes or less. In some embodiments, the method is performed as a single-step assay that provides a result in 15 minutes or less.

Also disclosed herein are systems for detection of one or more analytes in a sample comprising: a) one or more detection probes capable of specific binding or hybridization with the one or more analytes, wherein the one or more detection probes are conjugated to metal nanoparticles; and b) one or more nanostructured LSPR surfaces, wherein the one or more nanostructured LSPR surfaces are pre-functionalized with one or more primary binding components capable of specific binding or hybridization with the one or more analytes; wherein the plasmon resonance properties of the metal nanoparticles and those of the one or more nanostructured LSPR surface have been adjusted to substantially match in order to optimize detection sensitivity; and wherein the formation of bound complexes between the one or more detection probes, the one or more analytes, and the one or more primary binding components on the one or more nanostructured LSPR surfaces produces a detectable change in a physical property of light transmitted by or reflected from the one or more nanostructured LSPR surfaces. In some embodiments, the metal nanoparticles are selected from the group consisting of Au nanoparticles, Ag/Au core/shell nanoparticles, or hybrid magnetic/plasmonic nanoparticles. In some embodiments, the plasmon resonance properties of the Ag/Au core/shell nanoparticles have been adjusted by a method selected from the group consisting of changing the number of rinse steps used to rinse Ag core nanoparticles used to fabricate the Ag/Au core/shell nanoparticles, changing the size of Ag core nanoparticles used to fabricate the Ag/Au core/shell nanoparticles, changing the shape of Ag core nanoparticles used to fabricate the Ag/Au core/shell nanoparticles, changing the thickness of an Au shell used to fabricate the Ag/Au core/shell nanoparticles, and any combination thereof. In some embodiments, the plasmon resonance properties of the one or more nanostructured LSPR surface have been adjusted by a method selected from the group consisting of changing the choice of materials used to fabricate the LSPR surface, changing the dimensions of the layers of material used to fabricate the LSPR surface, changing the number of layers of material used to fabricate the LSPR surface, changing the order of the layers used to fabricate the LSPR surface, and any combination thereof. In some embodiments, the system further comprises one or more light sources for illuminating the one or more nanostructured LSPR surfaces. In some embodiments, the one or more light sources are selected from the group consisting of an LED, a halogen source, and a laser, or any combination thereof. In some embodiments, the system further comprises one or more detectors for detecting a physical property of light transmitted by or reflected from the one or more nanostructured LSPR surfaces. In some embodiments, the one or more detectors are selected from the group consisting of a photodiode, an avalanche photodiode, a photomultiplier tube, a CCD sensor, a CMOS sensor, an NMOS sensor, and any combination thereof. In some embodiments, the physical property of light is selected from the group consisting of intensity, spectrum, polarization, angle of reflection, and changes in RGB or greyscale value. In some embodiments, a limit of detection (LOD) for the method is better than 1 ug/mL. In some embodiments, a limit of detection (LOD) for the method is better than 1 ng/mL. In some embodiments, a limit of detection (LOD) for the method is better than 100 pg/mL. In some embodiments, a limit of detection (LOD) for the method is better than 10 pg/mL. In some embodiments, a limit of detection (LOD) for the method is better than 1 pg/mL. In some embodiments, a limit of detection (LOD) for the method is better than 0.1 pg/mL. In some embodiments, the system provides a detection result in 30 minutes or less. In some embodiments, the system provides a detection result in 15 minutes or less. In some embodiments, the detection result includes a determination of concentration of the one or more analytes. In some embodiments, the one or more pre-functionalized, nanostructured LSPR surfaces are packaged within a disposable fluidic device that further comprises fluidic components selected from the group including fluid channels, reaction wells, sample reservoirs, reagent reservoirs, and any combination thereof. In some embodiments, the disposable fluidic device interfaces with an instrument that comprises additional components selected from the group consisting of light sources, detectors, lenses, mirrors, filters, beam-splitters, prisms, polarizers, optical fibers, pumps, valves, microprocessors, computers, computer readable media, and any combination thereof. In some embodiments, the disposable fluidic device interfaces with a smartphone. In some embodiments, the disposable fluidic device interfaces with a mobile camera.

Disclosed herein are systems capable of detecting an analyte in a sample without the use of fluorophores or dyes, the system comprising: a) one or more detection probes capable of specific binding or hybridization with the one or more analytes, wherein the one or more detection probes are conjugated to nanoparticles; and b) one or more nanostructured LSPR surfaces, wherein the one or more nanostructured LSPR surfaces are pre-functionalized with one or more primary binding components capable of specific binding or hybridization with the one or more analytes; wherein the formation of bound complexes between the one or more detection probes, the one or more analytes, and the one or more primary binding components on the one or more nanostructured LSPR surfaces produces a detectable change in a physical property of light transmitted by or reflected from the one or more nanostructured LSPR surfaces; and wherein the limit-of-detection of the system is better than 100 pg/mL. In some embodiments, the nanoparticles are Au nanoparticles, Ag/Au core-shell nanoparticles, or hybrid nanoparticles having both a magnetic and plasmonic component. In some embodiments, the plasmon resonance properties of the Au nanoparticles, Ag/Au core/shell nanoparticles, or hybrid nanoparticles having both a magnetic and plasmonic component and those of the one or more nanostructured LSPR surface have been adjusted to substantially match in order to optimize detection sensitivity. In some embodiments, the analyte is alpha fetoprotein (AFP). In some embodiments, the detection result is provided in 30 minutes or less. In some embodiments, the detection result is provided in 15 minutes or less. In some embodiments, the detection is quantitative and the result comprises a determination of a concentration of the analyte.

Disclosed herein are kits comprising: a) the Ag/Au core/shell nanoparticles of claim 1; and b) reagents for use in conjugating the Ag/Au core/shell nanoparticles with primary or secondary binding components. In some embodiments, the primary or secondary binding components are selected from the group consisting of antibodies, antibody fragments, peptides, proteins, aptamers, oligonucleotides, and any combination thereof.

Also disclosed herein are kits for detection of an analyte in a sample, the kit comprising: a) A capture binding component that is specific for the analyte; and b) A detection binding component that is specific for the analyte, wherein the at least one detection binding component is conjugated to the Ag/Au core/shell nanoparticles of claim 1. In some embodiments, the capture and detection binding components are selected from the group consisting of antibodies, antibody fragments, peptides, proteins, aptamers, oligonucleotides, and any combination thereof. In some embodiments, the analyte is selected from the group consisting of a peptide, a protein, an oligonucleotide, a DNA molecule, an RNA molecule, a virus, a bacterium, a cell, a pathogen, a lipid molecule, a carbohydrate molecule, a small organic molecule, a drug molecule, an ion, creatinine, lactate, C-reactive protein (CRP), alpha-fetoprotein (AFP), cardiac troponin I (cTnI), cardiac troponin T (cTNT), cardiac phosphocreatine kinases M and B (CK-MB), brain natriuretic peptide (BNP), cortisol, S100BB, tau protein, thyroid-stimulating hormone (TSH), circulating tumor cells (CTC's), and any combination thereof. In some embodiments, the sample is selected from the group consisting of air, water, soil, a gas, an industrial process stream, feces, biological tissue, cells, blood, plasma, serum, sweat, tears, urine, cerebral spinal fluid, saliva, and any combination thereof.

Disclosed herein are kits for detection of an analyte in a sample, the kit comprising: a) A detection binding component that is specific for the analyte, wherein the detection binding component is conjugated to the Ag/Au core/shell nanoparticles of claim 1; and b) An LSPR sensor, wherein a sensor surface is conjugated with a capture binding component that is specific for the analyte. In some embodiments, the capture and detection binding components are selected from the group consisting of antibodies, antibody fragments, peptides, proteins, aptamers, oligonucleotides, and any combination thereof. In some embodiments, the analyte is selected from the group consisting of a peptide, a protein, an oligonucleotide, a DNA molecule, an RNA molecule, a virus, a bacterium, a cell, a pathogen, a lipid molecule, a carbohydrate molecule, a small organic molecule, a drug molecule, an ion, creatinine, lactate, C-reactive protein (CRP), alpha-fetoprotein (AFP), cardiac troponin I (cTnI), cardiac troponin T (cTNT), cardiac phosphocreatine kinases M and B (CK-MB), brain natriuretic peptide (BNP), cortisol, S100BB, tau protein, thyroid-stimulating hormone (TSH), circulating tumor cells (CTC's), and any combination thereof. In some embodiments, the sample is selected from the group consisting of air, water, soil, a gas, an industrial process stream, feces, biological tissue, cells, blood, plasma, serum, sweat, tears, urine, cerebral spinal fluid, saliva, and any combination thereof. In some embodiments, the plasmon resonance properties of the Ag/Au core/shell nanoparticles are adjusted by a method selected from the group consisting of changing the number of rinse steps used to rinse Ag core nanoparticles used to fabricate the Ag/Au core/shell nanoparticles, changing the size of Ag core nanoparticles used to fabricate the Ag/Au core/shell nanoparticles, changing the shape of Ag core nanoparticles used to fabricate the Ag/Au core/shell nanoparticles, changing the thickness of an Au shell used to fabricate the Ag/Au core/shell nanoparticles, and any combination thereof. In some embodiments, the plasmon resonance properties of the LSPR surface are adjusted by a method selected from the group consisting of changing the choice of materials used to fabricate the LSPR surface, changing the dimensions of the layers of material used to fabricate the LSPR surface, changing the number of layers of material used to fabricate the LSPR surface, changing the order of the layers used to fabricate the LSPR surface, and any combination thereof. In some embodiments, the LSPR sensor is packaged in a test strip or microfluidic device.

Disclosed herein are nanoparticle compositions comprising: (i) a magnetic component, and (ii) a plasmonic component. In some embodiments, the nanoparticle has a core/shell structure, and wherein the core is magnetic and the shell is plasmonic. In some embodiments, the nanoparticle has a core/shell structure, and where the core is plasmonic and the shell is magnetic. In some embodiments, the nanoparticle has a core/shell/shell structure, and wherein the core and the two shells each comprise a different material selected from the group consisting of a glass or polymer material, a magnetic material, and a plasmonic material. In some embodiments, a dimension of the plasmonic component ranges from about 20 nm to about 100 nm. In some embodiments, a dimension of the magnetic component ranges from about 50 nm to about 500 nm. In some embodiments, the magnetic component comprises a material selected from the group consisting of iron oxide, nickel, cobalt, a rare-earth-based magnetic material, or any combination thereof.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-D illustrate the mechanism underlying LSPR-based sensors. Adsorption of material that causes a change in local index-of-refraction or dielectric constant at the sensor surface (FIG. 1A) results in a shift of the plasmon absorption peak for light reflected from the sensor surface (FIG. 1B), which in turn may be monitored as a function of time to create sensorgrams (FIG. 1C) that indicate the kinetics for adsorption or binding events taking place at the sensor surface. In FIG. 1D, monitoring of the plasmon shift is performed using a digital camera (CCD or CMOS) and a set of focusing lenses so as to project an image of the sensor surface on the detector chip.

FIGS. 2A-B illustrate the principle underlying assays that utilize plasmon-plasmon coupling for LSPR sensor signal amplification. FIG. 2A: Assay using localized surface plasmon resonance (LSPR) films with a metal-dielectric-metal stack morphology and plasmonic nanoparticle probes, e.g. Au, Ag, or core/shell Ag/Au nanoparticles, or nanoparticle scaffolds having a magnetic component as illustrated in FIG. 6. The plasmonic nanoparticle probes are conjugated with antibodies specific to the antigen to be detected. The probes are mixed with the sample to be analyzed and added onto the LSPR biosensor. FIG. 2B: Probes are attached to the LSPR surfaces through an antigen bridge. The proximity of the probes to the surface induces a plasmon-plasmon coupling that causes a large shift in the surface plasmon peak position. The shift can be monitored in real time using any of the optical configurations described in FIG.1 or elsewhere in this disclosure. The limit of detection is <100 pg/mL in the case of cortisol, and <400 pg/mL for alpha fetoprotein (AFP).

FIGS. 3A-B illustrate the sensitivity improvement achieved by using plasmon-plasmon coupling to enhance LSPR signals. FIG. 3A: Data for a sequential sandwich assay performed using an anti-AFP capture antibody immobilized on the LSPR surface, a sample with various amounts of AFP (0, 37.5, 75, 150 ng/mL), and an anti-AFP detection antibody. The sample is incubated at 37° C. for 30 min, rinsed, and the detection antibody is added at room temperature at a concentration of 50 ug/mL. The reaction monitors the response of the detection antibody for 15 min. This 3-step assay lasts ˜50 to 60 minutes and generates an LSPR peak shift of <150 pm over the entire AFP concentration range of 1 to >300 ng/mL. FIG. 3B: For comparison, the same capture antibody is immobilized on the LSPR surface. Detection antibodies conjugated to gold nanoparticles (40 nm diameter, OD=1) are mixed with the AFP antigen sample (0, 37.5, 75, 150 ng/mL); immediately after addition of the gold nanoparticle-conjugated antibody-sample mixture, the plasmon peak position of the surface starts to shift due to the immobilization of the gold nanoparticles on the surface. Plasmon peak shifts that were ˜100 pm after 15 min in FIG. 3A are larger than 3000 pm in FIG. 3B.

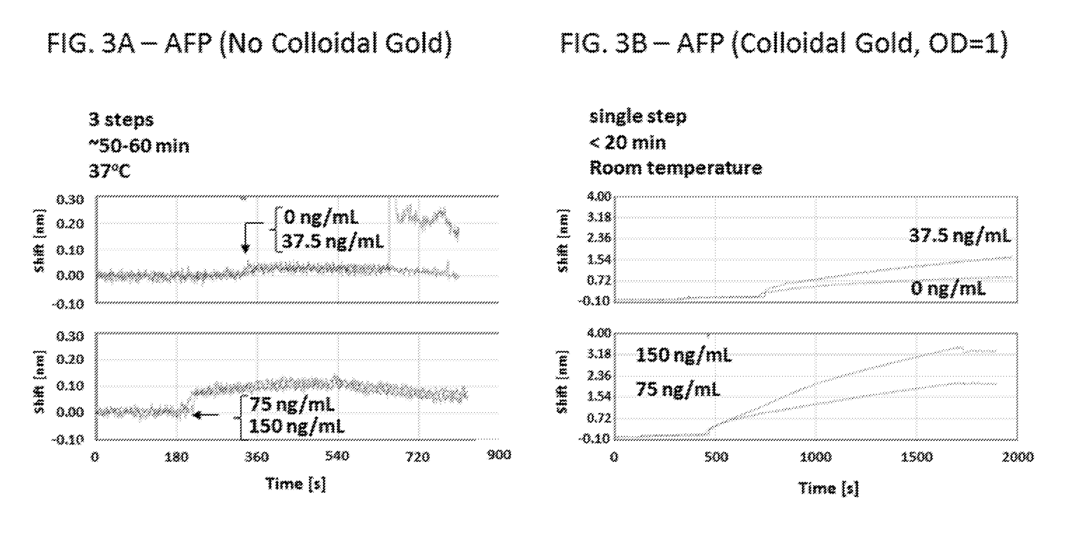

FIGS. 4A-B illustrate dose response curves for two model assay systems (alpha-fetoprotein (AFP) and cortisol) that indicate that the enhanced assay sensitivity achieved through plasmon-plasmon coupling is model independent. FIG. 4A: In case of AFP, concentrations of 1 ng/mL have been detected using anti-AFP detection antibodies conjugated to 40 nm Au nanoparticles. FIG. 4B: Cortisol at concentrations below ˜<100 pg/mL have been detected in a competitive assay using 40 nm gold colloids conjugated to cortisol. Note that these limits-of-detection can be improved by choosing different types of plasmonic nanoparticles (e.g. of different material type, particle shape, and particle size).

FIGS. 5A-B illustrate schematically the difference between an antibody-enzyme conjugate (FIG. 5B) and a conjugate composed of a nanoparticle scaffold (FIG. 5A). As described elsewhere in this disclosure, nanoparticle scaffold conjugates may have multiple antibody and/or enzyme molecules attached to the same nanoparticle, where the antibody and enzyme molecules may each be of the same type or may be a mixture of different antibodies and different enzymes.

FIGS. 6A-C illustrate schematically different types of nanoparticles having a dual magnetic and plasmonic property (not to scale). FIG. 6A: The dual-function nanoparticles may have a core/shell structure where the core is magnetic and the shell is plasmonic, or vice versa, where the core is plasmonic and the shell is magnetic. This includes also geometries where the shell is non-continuous, e.g. where a central core with either magnetic or plasmonic function is surrounded by multiple nanoparticles with the opposite function. FIG. 6B: The dual-function nanoparticles may have a dumbbell structure where the magnetic and plasmonic functions are contributed by different nanoparticles in a side-by-side geometry. FIG. 6C: More complex geometries like core/shell/shell are also possible where a third material such as glass or a polymer may serve as core or as a shell.

FIG. 7 shows an SEM image of core/shell Ag/Au nanoparticles synthesized following the method described hereafter. This is an example of particles with plasmonic properties. The Ag cores have triangular or hexagonal shapes consistent with a cubic close-packed crystal structure and with an approximate diameter of 30-50 nm. The cores are stabilized by a thin layer of gold. Energy dispersive X-ray (EDX) analysis (not shown) reveals the presence of Ag but not of Au. This is likely due to the fact that the gold shell is very thin (1-3 nm); this is consistent with a thin gold coating (of a few atomic layers) that stabilizes the Ag core particles against galvanic etching in salt solutions.

FIG. 8 shows a side-by-side comparison of the use of streptavidin conjugated to 40 nm gold nanoparticles and streptavidin conjugated to Ag/Au (core/shell) nanoparticles to detect binding on a biotinylated sensor surface. Biotinylated antibodies are immobilized on all LSPR surfaces. The sensor surfaces are probed with the streptavidin modified Au and Ag/Au nanoparticles. In the first step, a solution of streptavidin modified Ag/Au is added to the biochip producing an immediate strong response due to the binding of the Ag/Au-SA to the surface. A control experiment is performed by adding free biotin to the solution, thereby blocking the binding sites of Ag/Au-SA and preventing it from binding to the biotin molecules on the surface. After a sudden jump due to the color of the nanoparticles, the signal is flat and drops back to its initial value after a brief rinse (˜1600 sec). At around 2000 sec, a similar binding experiment is performed with streptavidin modified Au nanoparticles. Again, while the biotin pre-blocked Au-SA shows a flat response, the Au-SA exhibits an immediate response. Notice however that the response of Au-SA is about 1.9 nm after 15 min, while the response of Ag/Au-SA is above 4.5 nm. This indicates that Ag/Au nanoparticles provide enhanced signal amplification compared to Au nanoparticles.

FIGS. 9A-F provide a visual illustration of the stability of Ag/Au nanoparticle samples during growth of the Au shell and the process used to titrate the amount of HAuCl₄ required. In each image, the left tube contain the as-grown Ag/Au nanoparticles in water at a particular stage of Au shell growth, and the tube on the right contains the same as-grown Ag/Au nanoparticles in 166 mM NaCl. The amount of HAuCl₄ used increases going from FIG. 9A to 9F. Notice in FIG. 9A how the Ag/Au nanoparticles undergo considerable fading within a few minutes due to galvanic etching of the Ag cores by Na⁺ ions in solution. The degree of color fading decreases as the amount of HAuCl₄ used increases. When an Au shell protects the entire Ag core, the particles are no longer subject to galvanic etching and their color remain stable (FIGS. 9E and F).

FIG. 10 shows plasmon absorption spectra for Ag/Au nanoparticle samples heated at 98-100° C. for 90 min (light grey) and a non-heated reference sample (black). Analysis of the UV-Vis spectrum indicates that a 2.87 nm blue-shift occurs, but there is no evidence of peak broadening due to particle aggregation caused by the heating.

FIG. 11 shows a greyscale image of Au nanoparticles (top row) and Ag/Au nanoparticles (bottom row) for various percentages of glycerol and water. From left to right, the index of refraction is n=1.333, 1.340, 1.345, 1.353, 1.366, 1.384, 1.398 and 1.413 respectively.

FIGS. 12A-B show the plasmon absorption spectra associated with each well shown in the image of FIG. 11, and confirms the larger sensitivity of Ag/Au nanoparticles to index-of-refraction changes (FIG. 12B) compared to that for pure Au nanoparticles (FIG. 12A).

FIGS. 13A-C illustrate one method for quantifying coupling efficiencies for conjugating IgG molecules to Ag/Au nanoparticles by monitoring the plasmon shift of a reference sample (unconjugated Ag/Au nanoparticles; dark grey curves) and that for the IgG-Ag/Au nanoparticle sample (light grey curves) using different coupling strategies.

FIGS. 14A-F illustrate various configurations of nanostructured LSPR sensors for use with the disclosed nanoparticle compositions and methods. FIGS. 14A and B illustrate different embodiments of multiple LSPR sensors fabricated on a single substrate. FIG. 14C illustrates multiple LSPR sensors packaged in a test strip format. FIG. 14D illustrates an LSPR sensor chip packaged in a microfluidic device format. FIG. 14E illustrates an assay instrument system, where an LSPR sensor device interfaces with the instrument to provide optical detection, fluidics control, data acquisition, data storage, and data analysis capabilities. FIG. 14F illustrates the use of a smartphone to read the color change of an LSPR surface on a test card. The test card contains membrane-based fluidics or microfluidics to deliver the sample to the LSPR sensing location.

### DETAILED DESCRIPTION OF THE INVENTION

Disclosed herein are compositions, methods, devices, and systems for performing single-step, localized surface plasmon resonance (LSPR)-based plasmonic assays having exceptional assay sensitivity and extremely low limits of detection (LODs). The fabrication and use of Ag/Au core/shell nanoparticles are described, which may be used either in solution-based assays, in conjunction with conventional LSPR surfaces to develop biosensors, or with the nanostructured multi-stack LSPR surfaces also described herein to develop single-step, LSPR-based assays that exploit plasmon-plasmon coupling as a signal amplification mechanism for achieving high sensitivity and low limits of detection. The ability to tune the plasmon resonance properties of both the Ag/Au nanoparticles and those of the nanostructured multi-stack LSPR surface so that they substantially overlap, thereby allowing one to optimize plasmon peak shift and maximize assay sensitivity is one of the unique features of the presently disclosed compositions and methods. Another benefit of using the Ag/Au nanoparticles and nanostructured multi-stack LSPR surfaces of the present disclosure is that the short-range distance-dependence for plasmon-plasmon coupling may be exploited to develop single-step, homogeneous assays, e.g. assays where the initial molecular binding interaction takes place in solution, and that require no subsequent separation or rinse steps prior to detection using an LSPR sensor.

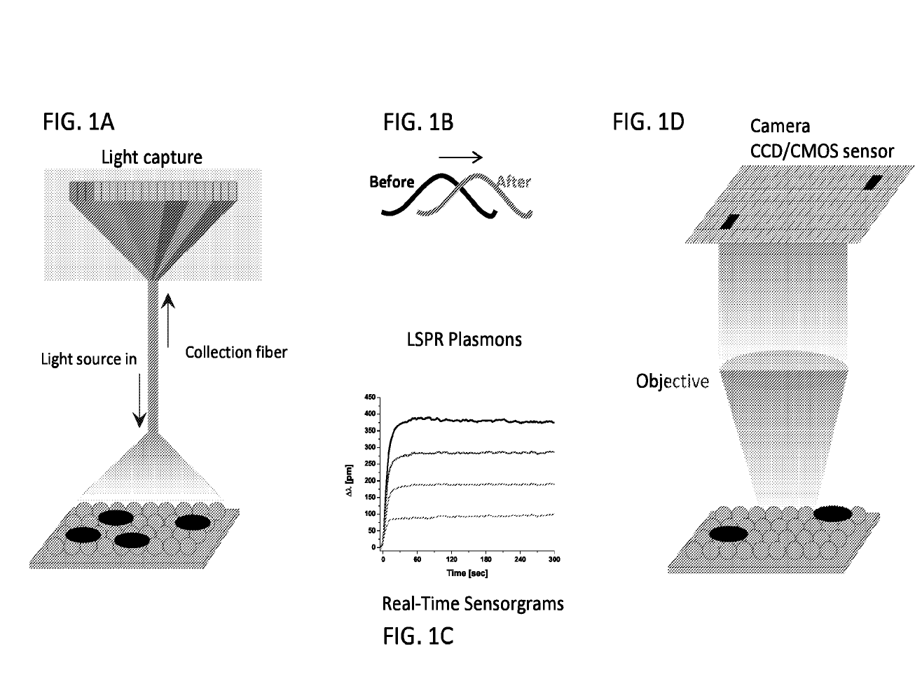

Overview of LSPR technology: Localized surface plasmon resonance (LSPR) sensors rely on the extreme sensitivity of the position of the surface plasmon absorption maximum to the local environment in the immediate vicinity of the interface. In particular, the signal transduction mechanism in LSPR sensors is often associated with a change in the index of refraction (or dielectric constant) near an LSPR-active surface (i.e. a surface capable of sustaining localized surface plasmons). The signal transduction mechanism in LSPR sensors may be associated with a change in an optical property of the sensor surface (e.g., shift in an absorption maximum for light) or a change in optical properties of light reflected from the LSPR-active surface. An LSPR-active surface may refer to an LSPR sensor surface. If an LSPR sensor surface is placed in contact with a film or solution of index of refraction n₁, followed by deposition on the surface of a material having an index of refraction n₂, the wavelength of the plasmon absorption maximum shifts by a value Δλ, as illustrated schematically in FIGS. 1A-C. It is possible to link the plasmon shift to the change in index of refraction Δn=n₂−n₁ through the following relation:

Δλ=m*Δn[1−e^{(-2L/δ)}] (1)

where m is a constant representing the sensitivity of the sensor, L is the thickness of the deposited material with index of refraction n₂, and δ is the decay length of the evanescent plasmon field. In addition to monitoring the shift in absorption maximum, in some cases, the change in index of refraction (or dielectric constant) near the sensor surface may be detected by monitoring other optical properties, for example, changes in reflection angle of the incident light, changes in the intensity of transmitted light, changes in the polarization of light reflected from the surface, changes in RGB or greyscale values of the reflected light (FIG. 1D), etc. The optical properties of the surface, or of light transmitted or reflected by the surface, may then be monitored using any of a variety of light sources and detectors as described further below.

Equation (1) was originally proposed for surface plasmon resonance (SPR) as an attempt to extract a quantitative measurement of the thickness or surface density of an adsorbed layer from the SPR response (see L. S. Jung, et al., Langmuir, 14, 5636-5648, 1998). Later, it was found that it can be applied to LSPR responses as well (see J. N. Anker, et al, Nature Materials, 7, 442-453, 2008). It describes the parameters that affect the sensor response without an explicit knowledge of the molecular mechanism responsible for the shift. A few general comments about Equation (1) will explain the need for a better understanding of the molecular mechanism responsible for the LSPR shift and the reasoning behind the current invention.

The observation that large proteins produce larger LSPR shifts than smaller proteins/molecules is explained using Equation (1) by the fact that the monolayer thickness L is larger for the former. Another particular prediction is that the overall plasmon shift Δλ, is capped at an upper value given by max Δλ=m*Δn. In fact, it has been observed that the maximum LSPR shift (max Δλ) is less than 5 to 10 nm for any known biomolecule on all gold-based LSPR colloids or nanostructured surfaces. For instance, measurements of the maximum shift produced by the binding of streptavidin to an LSPR surface are around ˜2 nanometers. This holds true if the binding is monitored on a LSPR gold biochip (surface area ˜mm²), on a single 40 nm gold bead (surface area ˜1600 nm²; see G. Raschke, et al., Nano Letters, 3, 935-938, 2003), or on a single gold nanorod (see C. L Baciu, et al., Nano Letters, 8, 1724-1728, 2008), i.e. on sensing surface areas spanning 9 orders of magnitude. Finally, when only a few biomolecules bind to the LSPR surface, they form a film with a sub-monolayer coverage. At the limit of very few binding events, the value of L in Equation (1) becomes arbitrarily small and the overall shift Δλ vanishes. This defines the limit of detection (LOD) of the technology and its analytical sensitivity. Empirically, the LOD of LSPR is marginally dependent on the nature of the antigen. For antigen molecules with molecular weight between 20 and 150 kDa, the LOD is in the range of 10-50 ng of antigen per milliliter of sample.

For application in the field of diagnostics, where concentrations of biomarkers fall in the sub-nanogram per ml range, the binding of biomarkers to an LSPR surface forms an exceedingly sparse sub-monolayer. According to Equation (1), the resulting LSPR shift becomes vanishingly small and falls below the limit of detection. It is common practice to enhance the signal using a secondary antibody, specific to the antigen thereby adding mass to the sensor surface (or slightly increasing the thickness of the sub-monolayer). However, even in this scenario, the additional mass provided by the secondary antibody does not result in detection for antigen at concentrations of a few tens of nanograms per ml. For detection in the realm of biomarker diagnostics, more mass should be added. In this regard, we have described in previous patent applications various ways to increase the overall LSPR response to render the LSPR biosensor more sensitive to the low end of antigen concentration in solution. For instance, in one patent (U.S. Pat. No. 8,426,152 B2) we have described an enzymatic assay for LSPR using a nanostructured LSPR films with a metal-dielectric-metal stack morphology. The additional mass comes from an enzymatic reaction that continuously converts a soluble moiety into an insoluble product that falls on the LSPR surface. In a second patent application (U.S. Patent Application Publication No. 2015/0247846 A1) we have disclosed a digital implementation of the detection methodology to reach lower detection limits using the same nanostructured LSPR films with a metal-dielectric-metal stack technology in an imaging mode.

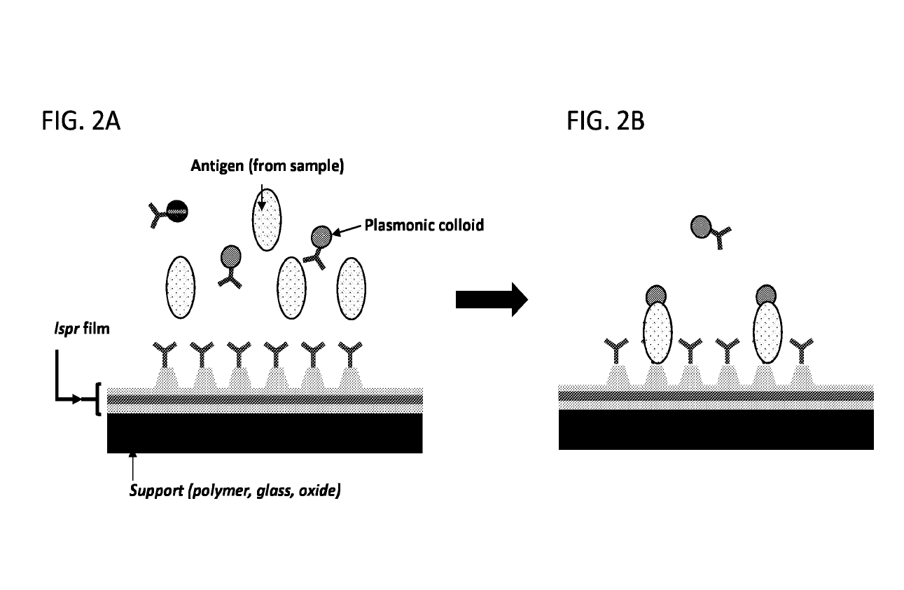

Overview of the present invention: Here we describe an additional method that relies on the use of nanoparticle plasmonic probes in conjunction with nanostructured LSPR films having a metal-dielectric-metal stack morphology to increase assay sensitivity to the sub-nanogram per ml range and to reduce the response time of the assay (see FIGS. 2A and B). The invention is rendered possible by a unique combination of plasmonic nanoparticles in solution and our particular LSPR film morphology as explained in the next paragraph.

The basic physical phenomenon responsible for Equation (1) is the dipolar interaction between the transient dipole moment of a biomolecule approaching the surface and the localized surface plasmon of the surface. To a first approximation, the magnitude of this interaction is proportional to the product of the corresponding polarizabilities: V_{dipolar}∝α_{biomolecule}·α(λ)_{LSPR surface}, where α_{biomolecule} is the polarizability of the biomolecule in solution, and α(λ)_{LSPR surface} is the frequency-dependent polarizability of the LSPR film. The wavelength shift experienced by the LSPR film (Equation (1)) increases with the strength of the dipolar interaction.

The polarizability of biomolecules scales roughly with the number of amino acid residues in the sequence. For instance, the amino acid Tryptophan has a polarizability of 23 Å³, while proteins such as insulin (˜5,000 Da), cytochrome C (12,000 D) and myoglobin (16,700 D) have a polarizability of 580 Å³, 1200 Å³, and 1700 Å³ respectively. By extrapolation, an antibody with MW of 150,000 D has a polarizability in the range of 10,000-15,000 Å³. The scaling of the polarizability with biomolecule size explains why, in label-free experiments, the shifts observed for large molecules are larger than shifts for small molecules. Increasing the polarizabilities of the biomolecule approaching the surface causes a stronger interaction with the surface resulting in an enhanced shift in the plasmon position. There is a limit, though, to the degree of amplification achievable due to the upper limit on polarizability of natural biomolecules or other polymer-based particles.

On the other hand, the polarizability α(λ) of metal particles scales with the volume of the particle and depends on the dielectric function of the metal according to the following formula:

$$α ( λ ) = 4 π {R}^{3} \frac{ɛ - {ɛ}_{m}}{ɛ + 2 {ɛ}_{m}} \qquad ( 2 )$$

where R is the particle radius, ε(λ) is the complex dielectric function of the metal and εₘ is the dielectric constant of the medium in which the particle is embedded. The factor (ε(λ)−εₘ)/(ε(λ)+2εₘ) is ˜2 for Au and ˜7 for Ag at their respective resonance frequency. Therefore a 40 nm gold particle (R=400 Å) has a polarizability in excess of ˜10⁹ Å³. This is more than 5 orders of magnitude larger than the polarizability of a large biomolecule or antibody. If a metal particle approaches another metal particle, their dipolar interaction leads to an unusually large optical response that manifests itself as a very large LSPR wavelength shift in their absorption/extinction maxima. This strategy, combined with the use of LSPR nanostructured thin films constitute the basis for the current invention.

The mechanism described above is often referred as plasmon-plasmon coupling. It has been proposed as the basis of a molecular ruler (C. Sonnichsen, et al., Nature Biotech, 23, 741-745, 2005) to measure the distance between a pair of gold particles. The mechanism has also been used to reveal the dynamics of DNA binding and cleavage by single EcoRV restriction enzymes (B. M. Reinhard, et al., PNAS 104, 2667-2672, 2007) between a single pair of Au or Ag nanoparticles deposited on a glass surface. In these studies, plasmon shifts in excess of 20 nm for 40 nm gold dimers, and larger than 100 nm for 40 nm Ag dimers, have been reported. These shifts far exceed the shifts induced by the binding of biomolecules, i.e. streptavidin, onto a single LSPR nanoparticle or an LSPR thin film surface (˜2-3 nm; see, e.g., G. Raschke et al., Nano Letters, 3, 935-938, 2003).

The use of plasmonic coupling between nanoparticles in solution has been the subject of a number of studies (K. Aslan, et al., JACS, 127, 12115-12121, 2005; K. Alsan, et al, Current Opinion in Chemical Biology, 9, 538-544, 2005) and a patent (C. D. Geddes, U.S. Pat. No. 8,101,424 B2). In the Geddes patent, the inventor used nanoparticles with diameters ranging from 10 to 40 nm for the detection of streptavidin in solution using biotinylated Au colloids. The inventor reports a LOD for the streptavidin assay using biotinylated colloids of ˜5 nM (or ˜250 ng/mL). The affinity of the biotin-streptavidin interaction is orders of magnitude larger than the affinity of an antigen-antibody interaction. The LOD of a technology relying on a biotin-streptavidin bridge to bring the colloids close together should be much lower than the LOD using an antibody-antigen pairing interaction. Therefore, it is expected that using plasmon-plasmon coupling between antibody-conjugated gold colloids in solution would lead to LOD larger than ˜100-250 ng/mL. Since the concentration of clinically-relevant biomarkers in blood is often in the range of ˜1 to 100 pg/mL, the use of pairs of plasmonic nanoparticles in solution appears to be of limited use in diagnostics.

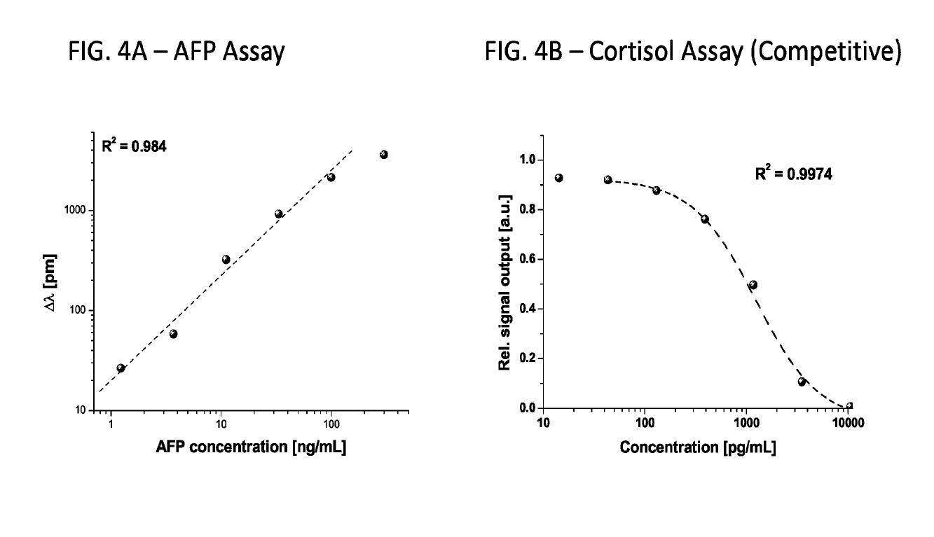

Disclosed herein is the combined use of nanostructured LSPR films with a metal dielectric-metal stack morphology (Takei, et al., U.S. Pat. No. 6,331,276 Al) and plasmonic nanoparticle reporters ranging in size from 20 to 100 nm to enhance the sensitivity of assays down to the 400 pg/mL range in 15 min for AFP and <100 pg/mL for cortisol (see FIGS. 4A-B), and potentially down to the sub-pg/mL range with further improvement.

The role of the nanostructured LSPR films with a metal-dielectric-metal stack morphology is crucial for proper signal amplification since these surfaces have an increased polarizability compared to metal particles. As a result, the interaction between a metal nanoparticle and the nanostructured metal-dielectric-metal LSPR surface is enhanced compared to the interaction between two metal nanoparticles in solution. In fact, the LSPR films with a metal-dielectric-metal stack morphology also exploit a plasmon-plasmon coupling mechanism between the base metal layer and the top metal layer to enhance its optical response. This is confirmed by the large absorption of the films for wavelengths in the range of 500-650 nm that gives the films their intense red/ruby color, while a similar amount of colloidal gold deposited on a glass surface (thereby lacking the base metal layer) produce films with only a pale pink coloration or even transparent. As a result, our invention provides amplification through the combined use of plasmonic probes in solution and the metal-dielectric-metal morphology of the LSPR film.

The model of plasmon-plasmon coupling also suggests different ways to optimize the coupling through the engineering of plasmonic nanoparticles of different size, shape, and materials, and through the manufacturing of LSPR surfaces with plasmon resonance peaks at different wavelengths. In particular, a resonance condition between the plasmon spectral properties of the nanoparticles and the plasmon spectral properties of the surface is expected to provide stronger coupling and enhanced sensitivity when applied to biological or chemical sensing applications.

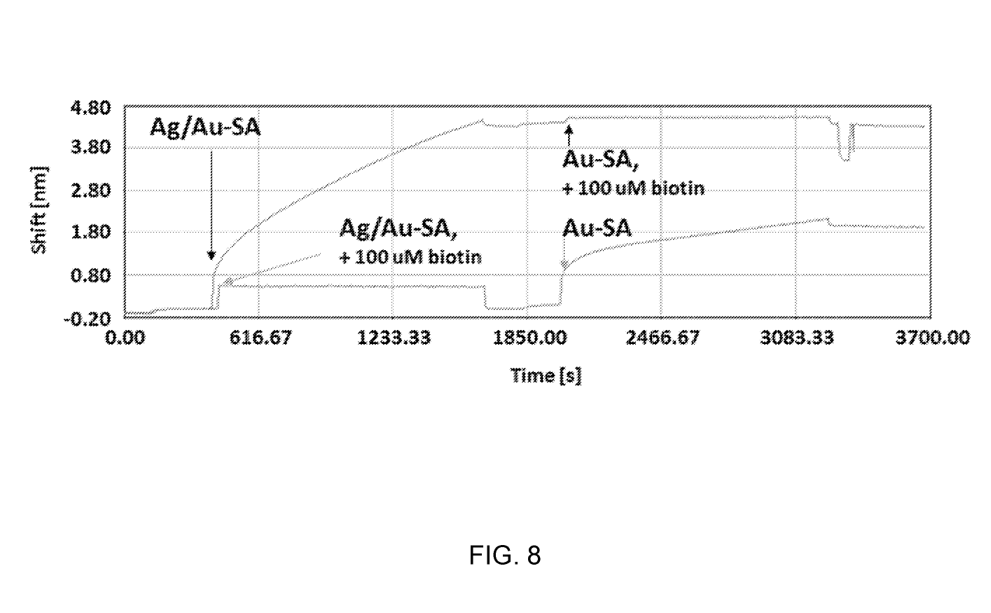

Amplification of the plasmon-plasmon coupling signal has been observed for both Au and Ag nanoparticles, with slightly higher amplification observed for the Ag nanoparticles (FIG. 8), as predicted by the theory described above. In order to exploit the plasmon-plasmon coupling phenomenon in developing high sensitivity assays, we have developed Ag/Au core/shell nanoparticles that provide both improved stability relative to Ag nanoparticles, and allow tuning of the plasmon resonance properties of the nanoparticles to match those of a nanostructured LSPR surface.

Synthesis of Ag nanoparticles and core/shell Ag/Au nanoparticles: Ag nanoparticles are synthesized at room temperature and in room light using a modification of protocols used to fabricate nanostructured metallic thin film surfaces and LSPR sensors. An important feature of the new procedure is the introduction of one or more specific polymers prior to the growth of the Au shell that serves to chemically stabilize the Ag core against galvanic etching.

In brief, a solution of silver nitrate (AgNO₃) in water is mixed with trisodium citrate and hydrogen peroxide. Silver ions (Ag⁺) in solution (provided by the AgNO₃) are reduced to metallic Ag^{o} by the rapid injection of sodium borohydride at room temperature. The initially transparent solution turns yellow colored after the injection. The color evolves in time to brown, then red, and finally blue as a result of the growth of the Ag nanoparticles. The reaction is left to proceed for about 30 minutes. At the end of the 30 minutes, the solution of Ag colloids is dark blue with a UV-Vis absorption peak above 700 nm.

The as-synthesized colloid solution is then centrifuged for 60 min at 13000×g. The supernatant is discarded, and the silver nanoparticles in the resulting pellet are then resuspended in double distilled water (ddH2O). The pellet consists of Ag nanoparticles. The resuspended Ag nanoparticles have a dark blue color. They are spun a second time at 13000×g and the pellet is resuspended in water. The UV-Vis absorption peak blue-shifts towards 650 nm. The process of washing the Ag nanoparticles causes the UV-Vis spectrum of the Ag plates to blue-shift. The washing process is thus repeated several times until the UV-Vis spectrum of the solution exhibits a sharp plasmon peak at around 450-480 nm (3-4 washes). In order to obtain a reproducible final product (silver nanoparticle core structures), it is critically important to thoroughly wash the Ag plates using a defined number of rinse steps to move the plasmon resonance peak to the desired wavelength.

The Ag nanoparticles (to be used as the core for Ag/Au core-shell nanoparticles) produced in this manner have stable plasmon absorption peaks in the 450-480 nm range, but are sensitive to the presence of salts in solution, e.g. NaCl, since metallic Ag^{o} can re-oxidize to form soluble Ag⁺ ions through the following redox reaction: Ag⁺(aq)+e⁻⇄Ag(solid), with a standard reduction potential of about +0.8 V. If not protected or stabilized, the Ag nanoparticles are entirely etched (dissolved) following the addition of any of several chemical species, including phosphine salts, NaCl, glycerol, or phosphate buffered saline (PBS).

To protect the Ag nanoparticles against galvanic etching and preserve their integrity, a thin shell of a chemically stable material is grown around the Ag nanoplates. Gold and silver share an identical crystal structure and their respective lattice parameter differs by only a few percent. Therefore, it is possible to grow a thin Au layer (or shell) on the Ag nanoparticles. The procedure used for the epitaxial growth of a gold shell is as follows. The plasmon peak of the starting Ag nanoparticle material is around 450-480 nm. It is shifted to about 530 nm by the addition of AgNO₃ in the presence of tri-sodium citrate. The Au shell is then grown by slowly adding a precise volume of an HAuCl₄ stock solution.

It is important to improve the chemical stability of the Ag core nanoparticles during growth of the Au shell. We have found that the use of polymers like poly-vinyl-pyrrolidone (PVP) and poly-vinyl-alcohol (PVA) of molecular weights in the range of 3,500 Da up to 50,000 Da work effectively. In general, the longer the length of the polymer used (i.e. the higher the molecular weight), the more stable the nanoparticles are to galvanic etching. However this is counter-balanced by the fact that a protective shell formed using a long polymer (higher molecular weight) yields a much reduced coupling efficiency for the subsequent conjugation of biomolecules to the Ag/Au nanoparticles. Hence a very precise and non-trivial titration of polymer molecular weight is required to establish optimal reproducibility and performance of the resulting nanoparticles.

In general, the molecular weight range for polymers used is about 3,500 Da to about 50,000 Da. In some embodiments, the molecular weight for the polymer used to stabilize the Ag core nanoparticles is at least 3,500 Da, at least 4,000 Da, at least 4,500 Da, at least 5,000 Da, at least 10,000 Da, at least 20,000 Da, at least 30,000 Da, at least 40,000 Da, or at least 50,000 Da. In some embodiments, the molecular weight of the polymer may be at most 50,000 Da, at most 40,000 Da, at most 30,000 Da, at most 20,000 Da, at most 10,000 Da, at most 5,000 Da, at most 4,500 Da, at most 4,000 Da, or at most 3,500 Da. The molecular weight of the polymer may have any value within this range, for example, about 12,000 Da. In some embodiments, the preferred polymer molecular weight range is about 1,000 Da to about 250,000 Da. In some embodiments, the range of polymer molecular weight is more preferably about 8,000 Da to about 30,000 Da.

In addition to PVA and PVP, other polymers that may be suitable for use in fabrication of the core/shell nanoparticles of the present invention include polymethylmethacrylate (PMMA), polyacrylic acid (PAA), cyclic olefin copolymers (COCs), polyacrylates, polyethylene glycols (or PEGs), various gums (Arabic, copal, spruce, and others), gelatin, or other polymers.

The optimal thickness of the polymer layer may range from about 1 nm to about 20 nm. In some embodiments, the thickness of the polymer layer may be at least 1 nm, at least 2, nm, at least 3 nm, at least 4 nm, at least 5 nm, at least 10 nm, or at least 20 nm. In some embodiments, the thickness of the polymer layer may be at most 20 nm, at most 10 nm, at most 5 nm, at most 4 nm, at most 3 nm, at most 2 nm, or at most 1 nm. Any of the lower and upper values described in this paragraph may be combined to form a range included within the disclosure, such as a polymer layer thickness ranging from about 3 nm to about 10 nm. In some embodiments, the preferred thickness of the polymer layer may range from about 2 nm to about 10 nm. The thickness of the polymer layer may have any value within this range, for example, about 2.5 nm thick.

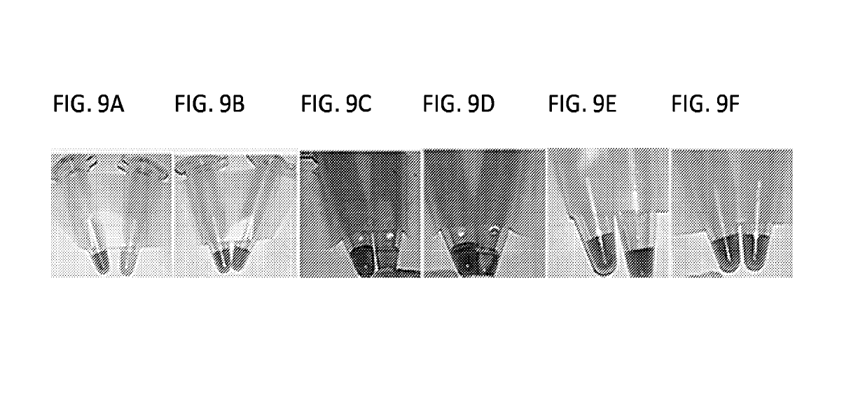

The amount of HAuCl₄ added is determined based on the chemical stability of the Ag/Au nanoparticles in 166 mM NaCl. If no Au layer is present, the addition of NaCl causes an immediate change in the color of the nanoparticle solution from red to transparent (see FIG. 9A). As the amount of gold precursor is slowly added, the color differential becomes less pronounced and eventually disappears when an optimal amount of Au is formed to fully protect the Ag plates (FIGS. 9B-F). At the end of the Au shell growth process, the final Ag/Au nanoparticle solution is thoroughly washed in milli-Q water. Typical ODs for the resultant samples are in the range of 5-20.

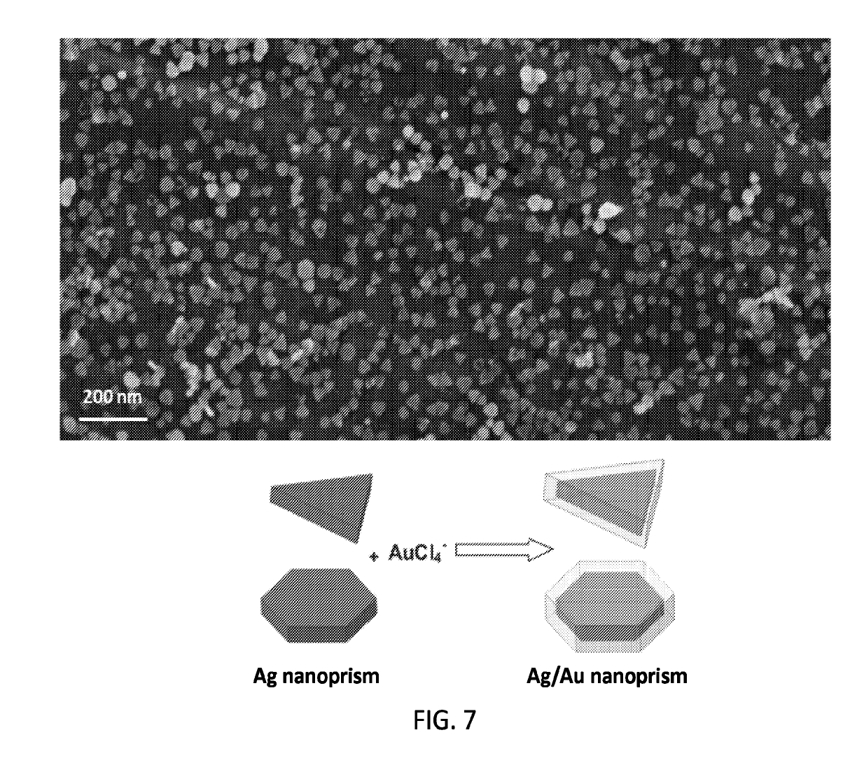

Size and shape of Ag/Au core/shell nanoparticles: Depending on the ratio of materials and conditions used for growth, the Ag/Au core/shell nanoparticles of the present disclosure may be of a variety of sizes and shapes. For example, the particles may be spherical, non-spherical cubic, cuboid, pyramidal, cylindrical, conical, oblong, star-shaped, in the form of short nanowires, hollow, porous, and the like. In some embodiments, the particles are of a triangular plate shape or a hexagonal plate shape having a long axis of about 30 nm to 100 nm, and a thickness of about 5 nm to about 10 nm. FIG. 7 shows an SEM image of core/shell Ag/Au nanoparticles synthesized as described herein.

In general, the nanoparticles have average dimensions ranging from about 5 to about 500 nanometers. In some embodiments, the nanoparticles may have average dimensions of at least 5 nm, at least 10 nm, at least 20 nm, at least 30 nm, at least 40 nm, at least 50 nm, at least 60 nm, at least 70 nm, at least 80 nm, at least 90 nm, at least 100 nm, at least 200 nm, at least 300 nm, at least 400 nm, or at least 500 nm. In some embodiments, the nanoparticles may have average dimensions of at most 500 nm, at most 400 nm, at most 300 nm, at most 200 nm, at most 100 nm, at most 90 nm, at most 80 nm, at most 70 nm, at most 60 nm, at most 50 nm, at most 40 nm, at most 30 nm, at most 20 nm, at most 10 nm, or at most 5 nm. In some embodiments, the nanoparticles may have average dimensions ranging from about 20 nm to about 80 nm. In preferred embodiments, the nanoparticles may have average dimensions ranging from about 40 nm to about 60 nm. Any of the lower and upper values described in this paragraph may be combined to form a range included within the disclosure, such as an average nanoparticle dimension ranging from about 10 nm to about 100 nm. Those of skill in the art will recognize that the average nanoparticle dimension may have any value with the above ranges, for example, about 44 nm.

These dimensions are primarily a result of the dimensions of the Ag core, as the gold shell is extremely thin, e.g. on the order of 1 and 20 atomic layers in thickness. In some embodiments, the gold shell comprises at least 1 atomic layer, at least 2 atomic layers, at least 3 atomic layers, at least 4 atomic layers, at least 5 atomic layers, at least 6 atomic layers, at least 7 atomic layers, at least 8 atomic layers, at least 9 atomic layers, at least 10 atomic layers, at least 11 atomic layers, at least 12 atomic layers, at least 13 atomic layers, at least 14 atomic layers, at least 15 atomic layers, at least 16 atomic layers, at least 17 atomic layers, at least 18 atomic layers, at least 9 atomic layers, or at least 20 atomic layers. In some embodiments, the gold shell comprises at most 20 atomic layer, at most 19 atomic layers, at most 18 atomic layers, at most 17 atomic layers, at most 16 atomic layers, at most 15 atomic layers, at most 14 atomic layers, at most 13 atomic layers, at most 12 atomic layers, at most 11 atomic layers, at most 10 atomic layers, at most 9 atomic layers, at most 8 atomic layers, at most 7 atomic layers, at most 6 atomic layers, at most 5 atomic layers, at most 4 atomic layers, at most 3 atomic layers, at most 2 atomic layers, or at most 1 atomic layer. Any of the lower and upper values described in this paragraph may be combined to form a range included within the disclosure, such as a number of atomic layers ranging from about 3 to about 11 atomic layers. Those of skill in the art will recognize that the number of atomic layers may have any value within the above range, for example, 7 atomic layers. An SEM of a typical Ag colloid sample is shown in FIG. 8.

Aggregates or assemblies of Ag/Au core/shell nanoparticles: In some embodiments of the disclosed compositions and methods, two or more Ag/Au core/shell nanoparticles may be cross-linked using suitable conjugation chemistries, or encapsulated in another material, e.g. a polymer, to create assemblies (e.g. aggregates, clusters, or conglomerates) of Ag/Au nanoparticles.

Stability testing of Ag/Au core/shell nanoparticles: The Ag/Au nanoparticle samples are tested for chemical stability in various buffers and at high temperatures by monitoring the plasmon peak wavelength as a function of time over a period of 2 days; the buffers used for testing are listed in Table 1. UV-Vis spectra are measured with a SpectroMax Pro 3401PC plate reader from 400 nm to 750 nm, with a step size of 1 nm. The peak position is computed using a proprietary algorithm. The buffers used are: Buffer 1: H₂O, 0.01% Tween 20; Buffer 2: PBS, pH 7.4, 0.01% Tween 20; Buffer 3: 40 mM Tris, 100 mM borate, 150 mM NaCl, 0.01% Tween 20, pH 6.98; Buffer 4: 20 mM HEPBS, pH 7.2-0.01% Tween 20. We have empirically observed that if the plasmon peak position is stable over this time period (40 hrs), it will typically remain stable for at least 6 months.

TABLE 1
Stability testing of Ag/Au core/shell nanoparticles in different buffers (peak
position in nm).

| | | 5 min | 48 min | 90 min | 240 min | 16 hrs | 40 hrs | Status |
|---|---|---|---|---|---|---|---|---|
| A | Buffer 1 | 535.68 | 535.21 | 535.18 | 534.91 | 535.28 | 535.10 | stable |
| B | Buffer 2 | 533.81 | 534.46 | 534.13 | 534.11 | 534.07 | 534.10 | stable |
| C | Buffer 3 | 534.29 | 534.62 | 534.68 | 534.74 | 535.76 | 535.10 | stable |
| D | Buffer 4 | 534.64 | 534.79 | 534.89 | 534.52 | 534.96 | 534.80 | stable |

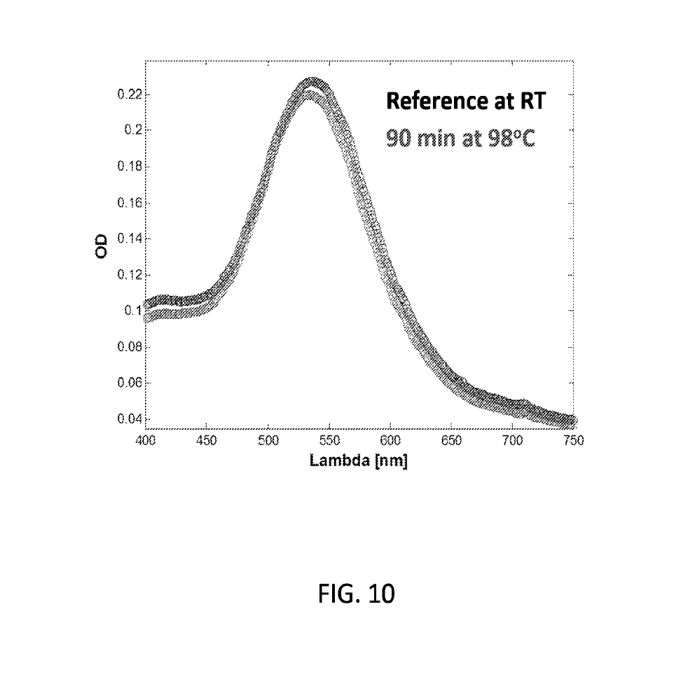

To further test their stability, samples of Ag/Au nanoparticles are heated to 95-100° C. for 90 minutes using a thermal block. After cooling back down to room temperature, a plasmon absorption spectrum of the heated sample is compared to the plasmon absorption spectrum of a non-heated reference sample (FIG. 10). We typically observe an insignificant blue-shift of the plasmon peak for the heated sample (˜1-2 nm). More importantly, there is no broadening of the plasmon peak spectra that could indicate particle aggregation.

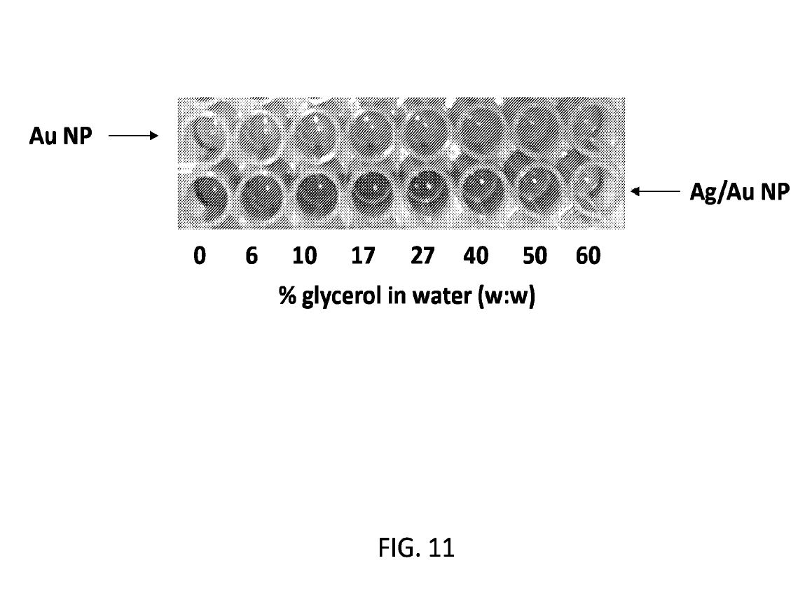

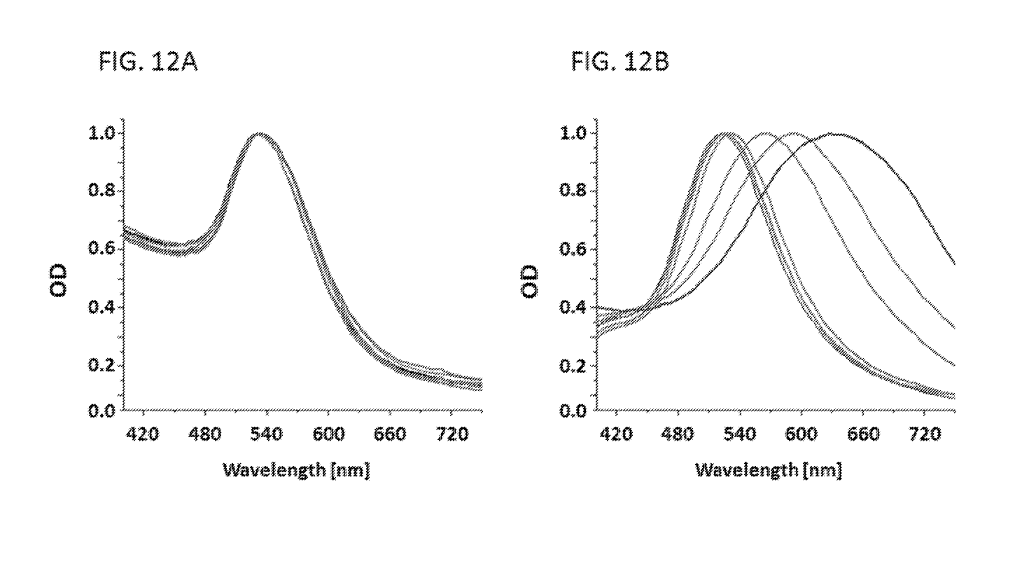

Sensitivity performance of Ag/Au nanoparticles versus 40 nm Au nanoparticles: The core/shell Ag/Au nanoparticles fabricated as described above are chemically-stable. Their optical properties are largely dominated by the Ag core (the thin Au shell has a marginal impact). To observe the response of the Ag/Au nanoparticles to changes in index of refraction, we have prepared mixtures of glycerol in water at different percentages (w:w) that resulted in homogenous solutions with tabulated indices of refraction. In this way, we can compare side-by-side the responses to a change in index of refraction for commercial 40 nm Au nanoparticles with those for the Ag/Au nanoparticles of the present disclosure. FIG. 11 shows a greyscale image of Au nanoparticles (top row) and Ag/Au nanoparticles (bottom row) for various percentages of glycerol and water. From left to right, the index of refraction is n=1.333, 1.340, 1.345, 1.353, 1.366, 1.384, 1.398 and 1.413 respectively. FIGS. 12A and B show the spectra associated with each well, and confirm that the Ag/Au nanoparticles exhibit higher sensitivity to changes of index of refraction (FIG. 12B) compared to the pure Au nanoparticles (FIG. 12A). The plasmon peak shift is linearly dependent on the change of index of refraction. The slope of the linear dependence (m=Δλ/Δn) is often taken as an indicator for the intrinsic sensitivity of a plasmonic probe. The data of FIGS. 12A-B show m_{Au}=35-40 nm/RI, and m_{Ag/Au}=430-690 nm/RI depending on the specific sample. The Ag/Au nanoparticles are 12 to ˜20 fold more sensitive than the 40 nm Au nanoparticles for index of refraction sensing.

A key feature of the disclosed Ag/Au core/shell nanoparticle compositions and methods disclosed herein is the ability to adjust the plasmon resonance properties of the nanoparticles to substantially match those of an LSPR surface when used in the development of biosensors. The plasmon resonance properties of the Ag/Au core/shell nanoparticles may be adjusted in a variety of ways, for example, by changing the number of rinse steps during the wash of the Ag core nanoparticles, by changing the size of Ag core nanoparticles, by changing the shape of Ag core nanoparticles, or by changing the thickness of an Au shell used to fabricate the Ag/Au core/shell nanoparticles.

Conjugation of biomolecules to Ag/Au nanoparticles: Adsorption of biomolecules to the Ag/Au nanoparticles through electrostatic interaction results in unstable conjugates in buffers of high ionic strength. It is therefore necessary to cross-link biomolecules to the surface of the nanoparticles using high-affinity or covalent cross-linking strategies to create a layer of immobilized biomolecules (i.e. a biomolecular layer). Since the surface of the nanoparticles is Au, we have developed a cross-linking strategy that uses mercapto groups to bind to the Au surface, and a secondary functional group capable of reacting with the biomolecule of choice. Variations of this strategy have been successfully implemented to prepare the Ag/Au nanoparticles for bioconjugation. For instance, the Ag/Au nanoparticles can be modified with mono- or di-thiol molecules possessing a secondary moiety such as a carboxyl, a hydrazine, a hydrizide, an amine, an aldehyde, a biotin, or any other functional group that can be chemically linked to the biomolecules. The chemical linkage between the two functional groups (the mono- or di-thiol group and the secondary moiety) is provided by a linker chain of variable length and variable composition. For instance, the linker chain could be an alkyl group comprised of N repetitive units, where N=2 to 20, or it could be a PEG (polyethylene glycol) linker comprising 4, 8, 12, 16, or more repetitive units. The linker chain can also contain aromatic rings, such as cyclohexane. We have been able to conjugate Ag/Au nanoparticles to human IgG antibodies using the cross-linking strategies described above.

One non-limiting example of a conjugation protocol used with the Ag/Au nanoparticles disclosed herein is as follows. Ag/Au nanoparticle solutions at OD=10 in water are modified with 0.2 mM of mercapto-undecanoic acid [SH—(CH₂)₁₀—COOH] for 2 hrs. After a wash step, the functionalized Ag/Au nanoparticles are incubated in 100 mM 2-(N-morpholino)ethanesulfonic acid (MES) with 100 mM 1-ethyl-3-(3-dimethylaminopropyl)-carbodiimide (EDC) and 5 mM N-hydroxysuccinimide (NHS) in the presence of an antibody or other biomolecule. After 2 hrs of incubation, the bioconjugate is washed twice in phosphate buffered saline (PBS) by pelleting the nanoparticles and resuspending them in PBS. The final solution is stored at 4° C. for further use. Other strategies for bioconjugation, for example, as described in Hermanson (G. T. Hermanson, Bioconjugates Techniques, 2^{nd} Edition, Academic Press, 2008), can also be used. For instance, glycosylated antibodies can be periodate-oxidized and reacted with a hydrazine-modified Ag/Au nanoparticle solution. The stability of the resulting conjugate is then further enhanced by reduction of the Schiff bond with sodium cyanoborohydride.

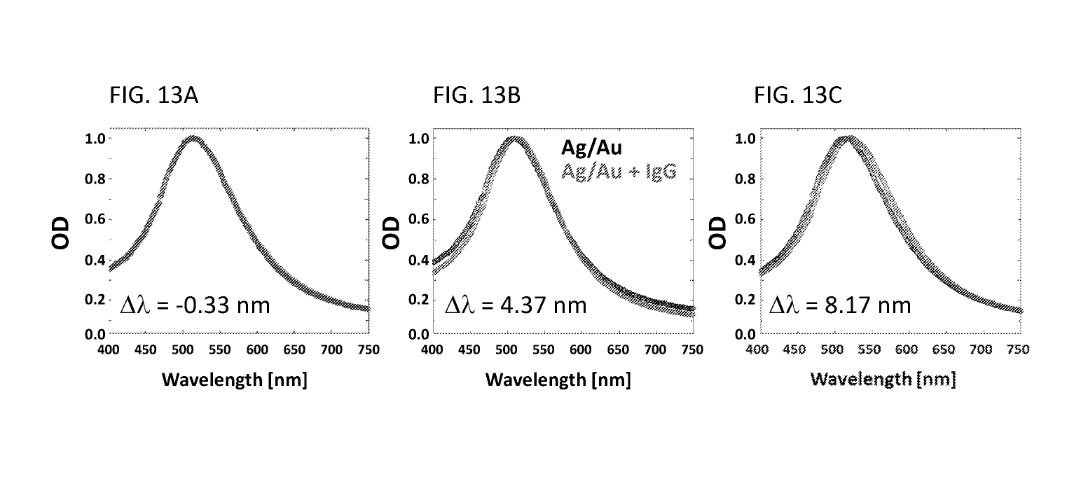

Coupling success can be evaluated by monitoring the plasmon peak position of the Ag/Au nanoparticle in solution before and after coupling (FIGS.13A-C). If the antibody is coupled to the Ag/Au nanoparticle, the plasmon peak should red-shift. The amount of red-shifting is proportional to the amount of biomolecule covalently bound to the Ag/Au nanoparticles. Spectra are recorded using a SpectroMax Pro 3401PC plate reader with spectral resolution of 1 nm. When IgG and Ag/Au nanoparticles are mixed in solution in the absence of a cross-linker, no significant difference in the plasmon absorption peak is observed indicating that passive adsorption does not occur (FIG. 13A). FIG. 13B illustrates coupling of the antibody to Ag/Au nanoparticles using EDC/NHS chemistry. FIG. 13C illustrates coupling of IgG to Ag/Au nanoparticles using hydrazine-aldehyde coupling. In the latter two cases, significant plasmon peak shifts of ˜4 nm and 8 nm respectively are observed, and indicate successful covalent coupling of the antibodies to the Ag/Au nanoparticles. Typical plasmon peak shifts observed after the conjugation reaction is complete range from 2 nm to 12 nm.

In all nanoparticle/bead-based assays using a capture surface, the analytical sensitivity is dependent on three factors:

1. The capture of the antigen by nanoparticles/beads in solution

2. The rate of transport of the antigen-nanoparticle/bead complexes towards the LSPR sensor
surface; and

3. The strength of the readout signal provided by the nanoparticles/beads when immobilized
on the sensing surface. This signal may be from fluorescence, phosphorescence, chemiluminescence, radiative decay, MRI, electrochemical, colorimeteric, etc.)

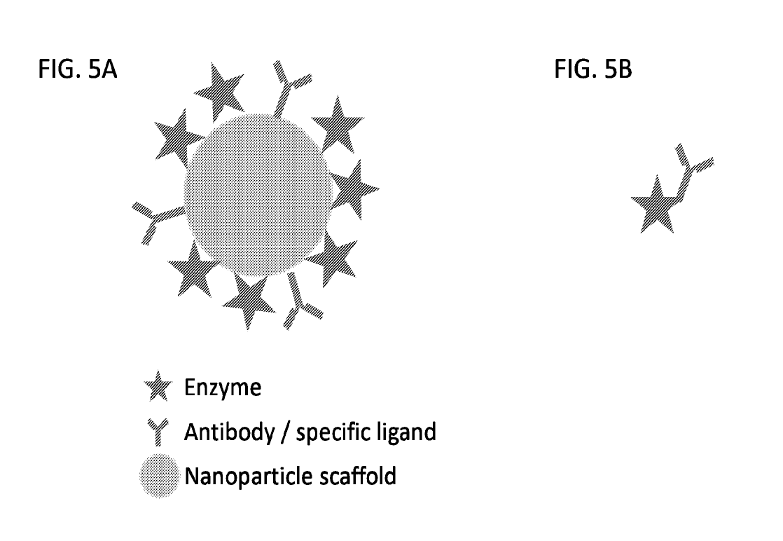

Nanoparticles conjugated to both antibodies and enzymes: Due to their large surface area compared to single antibodies, nanoparticle- or bead-antibody conjugates can capture more antigen from the sample than single antibody molecules (FIGS. 5A-B). Furthermore, the use of nanoparticles/beads suggests an additional route to improving the strength of the signal readout. Nanoparticles provide a scaffold that can be modified with multiple biological molecules having orthogonal functionality (FIG. 5A). One of the molecules could be an antibody, thereby conferring a binding specificity to the nanoparticles/beads. A second molecule could be an enzyme such as alkaline phosphatase (AP) or horse-radish peroxidase (HRP) that is used in a signal amplification mechanism, as in enzyme-linked immunoassays (ELISA). A simple estimation indicates that 20 to 30 proteins or biomolecules can be packed on a 40 nm colloid. For example, it should be possible to synthesize a 40 nm Au colloid functionalized with approximately 5 antibody molecules and 15 to 25 enzyme molecules. When immobilized on a surface due to the presence of an antigen in the sample, these enzyme-enriched nanoparticle conjugates will provide signal amplification enhanced by a factor of 10 or more compared to a commonly used antibody conjugated to a single enzyme.

We have described the coupling of antibody-enzyme conjugates and an LSPR surface in a recent patent as a way to improve the detection limits of an assay (U.S. Pat. No. 8,426,152 B2, Enzymatic Assay for LSPR). The use of a nanoparticle/bead coupled to several antibody and enzyme molecules would improve on the speed and sensitivity of the LSPR assay described in the '152 patent.

Hybrid magnetic/plasmonic nanoparticles: Au core or Ag/Au core/shell nanoparticles functionalized with multiple ligands and/or enzymes diffuse passively to the LSPR surface. Diffusion to the surface is the rate limiting step in surface-based assays. To increase the transfer rate from the solution to the surface, an external force needs to be applied. One popular approach is to use pulsed magnetic field gradients to transport particles to the LSPR sensor surface. This mechanism can be implemented using, for example, superparamagnetic (SP) beads of 200 nm to 500 nm in diameter. SP beads are mostly iron oxide (Fe₂O₃ & Fe₃O₄) colloids coated with a polymeric shell. As such, SP beads lack the strong polarizability needed to enhance the plasmonic response of a LSPR surface. However, it should be possible to synthesize a hybrid particle that has both magnetic and plasmonic properties. In this case, a magnetic field gradient can be used to manipulate (e.g. attract or repulse) the particles to or from the LSPR surface, while the plasmonic component imparts the large polarizability required to enhance the LSPR signal.

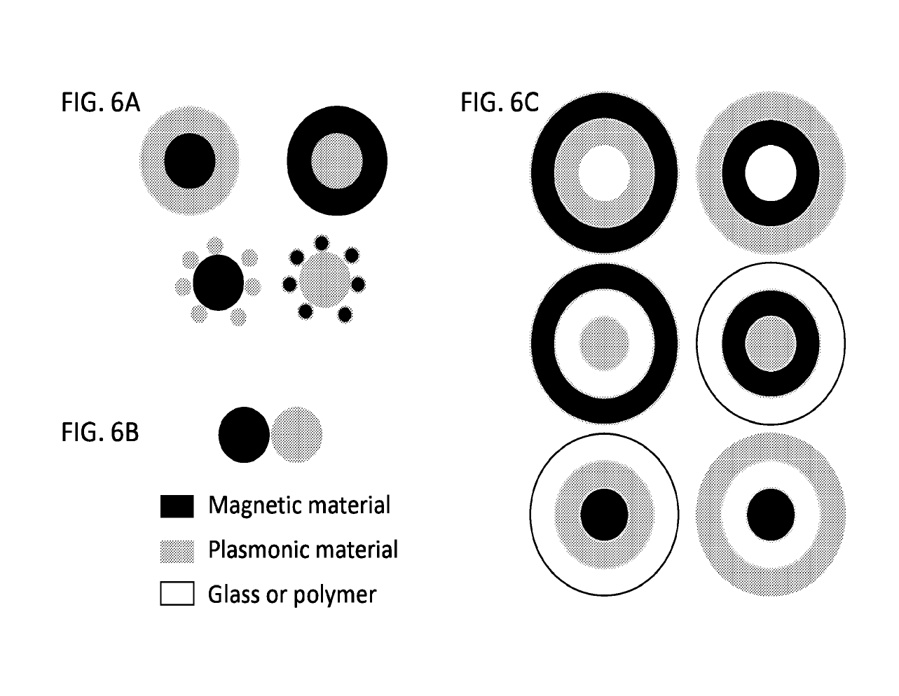

In some embodiments, the hybrid particles may have a core/shell structure (e.g. magnetic/plasmonic or plasmonic/magnetic). In some embodiments, the hybrid particles may have a core/shell/shell structure where a glass or polymer core is coated with a first magnetic shell surrounded by a second plasmonic shell, or vice versa. In some embodiments, the hybrid particles may have more than two shell layers in addition to the core, e.g. three shell layers, four shell layers, five shell layers, six shell layers, or more, which may comprise any combination of magnetic materials, plasmonic materials, polymer materials, dielectric materials, etc. In general, the core may be made of a glass, polymeric, dielectric, magnetic, or plasmonic material. Similarly, the one or more shells may be made of a glass, polymeric, dielectric, magnetic or plasmonic material. In general, the hybrid particles may comprise a core and one or more shell layers composed of any combination of these materials that yields a particle having both magnetic and plasmonic properties. Non-limiting examples of other possible hybrid nanoparticle geometries are illustrated in FIGS. 6A-C.

Materials for fabricating the magnetic components of hybrid magnetic/plasmonic nanoparticles/beads include, but are not limited to, iron oxides, cobalt, nickel, gadolinium (Gd) and Gd alloys, and more generally, magnetic particles and materials containing rare earth elements (neodymium (Nd), dysprosium (Dy), terbium (Tb), etc.). In general, materials known to be ferromagnetic, or exhibiting helical magnetic domains, are potentially applicable to the fabrication of hybrid magnetic/plasmonic nanoparticles, as are materials known to exhibit remanent magnetization (residual magnetism) or a spontaneous magnetization.

In one embodiment, a dimension of the magnetic component (e.g. the core diameter or a shell layer thickness) of the hybrid magnetic/plasmonic nanoparticles/beads may range from about 20 nm to about 1000 nm. In another embodiment, a dimension of the magnetic component of the hybrid magnetic/plasmonic nanoparticles/beads may range from about 100 nm to about 500 nm. In some embodiments, a dimension of the magnetic component may be at least about 20 nm, at least about 50 nm, at least about 100 nm, at least about 200 nm, at least about 300 nm, at least about 400 nm, at least about 500 nm, at least about 600 nm, at least about 700 nm, at least about 800 nm, at least about 900 nm, or at least about 1000 nm. In some embodiments, a dimension of the magnetic component may be at most about 1000 nm, at most about 900 nm, at most about 800 nm, at most about 700 nm, at most about 600 nm, at most about 500 nm, at most about 400 nm, at most about 300 nm, at most about 200 nm, at most about 100 nm, at most about 50 nm, or at most about 20 nm. Any of the lower and upper values described in this paragraph may be combined to form a range included within the disclosure, such as a dimension of the magnetic component ranging from about 50 nm to about 500 nm. Those of skill in the art will recognize that a dimension of the magnetic component of the hybrid magnetic/plasmonic nanoparticle/bead may have any value within this range, e.g. about 125 nm. In general, the dimension of the magnetic component (e.g. the diameter of a magnetic core or the thickness of a magnetic shell) is such that it provides a coupling to the external magnetic field gradient that is large enough to overcome Brownian motion (thermal fluctuations). With the magnetic field gradients currently available, the dimensions of the magnetic core or shell structures should be in the 100-500 nm range. In general, these dimensions will scale with the magnitude of the magnetic field gradient.

Materials for fabricating the plasmonic components of hybrid magnetic/plasmonic nanoparticles/ beads include, but are not limited to, noble metals such as gold, silver, platinum, palladium, and the like. In some embodiments, other metals, e.g. copper, may be used.

In one embodiment, a dimension of the plasmonic component (e.g. the core diameter or a shell layer thickness) of the hybrid magnetic/plasmonic nanoparticles/beads may range from about 20 nm to about 1000 nm. In another embodiment, a dimension of the plasmonic component of the hybrid magnetic/plasmonic nanoparticles/beads may range from about 100 nm to about 500 nm. In some embodiments, a dimension of the plasmonic component may be at least about 20 nm, at least about 50 nm, at least about 100 nm, at least about 200 nm, at least about 300 nm, at least about 400 nm, at least about 500 nm, at least about 600 nm, at least about 700 nm, at least about 800 nm, at least about 900 nm, or at least about 1000 nm. In some embodiments, a dimension of the plasmonic component may be at most about 1000 nm, at most about 900 nm, at most about 800 nm, at most about 700 nm, at most about 600 nm, at most about 500 nm, at most about 400 nm, at most about 300 nm, at most about 200 nm, at most about 100 nm, at most about 50 nm, or at most about 20 nm. Any of the lower and upper values described in this paragraph may be combined to form a range included within the disclosure, such as a dimension of the plasmonic component ranging from about 20 nm to about 100 nm. Those of skill in the art will recognize that a dimension of the plasmonic component of the hybrid magnetic/plasmonic nanoparticle/bead may have any value within this range, e.g. about 35 nm.

Nanostructured LSPR surfaces: A variety of methods may be used for fabricating nanostructured surfaces capable of sustaining localized surface plasmons, see for example, Takei, et al., U.S. Pat. No. 6,331,276, which is incorporated in its entirety herein. The components required to fabricate a nanostructured LSPR sensor may include substrates, metal layers or films, nanoparticles or nanostructures, and/or other dielectric or insulating materials. The plasmon resonance properties of the LSPR sensor surface may be adjusted by manipulating the choice of materials, the number and ordering of layers, and the thickness of the layers used to fabricate the sensor.

Sensor substrates: Nanostructured LSPR sensors may be fabricated using a variety of materials, including, but not limited to, glass, fused-silica, silicon, ceramic, metal, or a polymer material. In some embodiments, it is desirable for the substrate material to be optically transparent so that the sensor surface may be illuminated from the back side. In other embodiments, the sensor surface is illuminated from the front side, and the transparency or opacity of the substrate material is not important. In general, the substrates used for fabricating nanostructured LSPR sensors will have at least one flat surface, however, in some embodiments, the substrate may have a curved surface, e.g. a convex surface or a concave surface, or a surface of some other geometry.

Metal layers or films: In general, nanostructured LSPR sensors will comprise one or more metal layers or metallic thin films. In some embodiments, there may be about 1, 2, 5, 10, 15, 20, or more metal layers. In some embodiments, the preferred metal for use in layers or films will be noble metals such as gold, silver, platinum, palladium, and the like. In some embodiments, other metals, e.g. copper, may be used. The advantage of using a noble metal is their ability to support surface plasmon activity due to the high mobility of conductance band electrons. For some noble metals, an additional advantage is their ability to resist chemical corrosion or oxidation. The metal layers or metallic thin films may comprise any mixture and/or any combination of the preferred metals mentioned herein. For example, the metal layer may comprise of one layer of gold, one layer of copper, and one layer of a mixture of silver and platinum. Metal layers or films may be fabricated by any of the techniques known to those of skill in the art, including, but not limited to, thermal, electroplating, sputter coating, chemical vapor deposition, vacuum deposition, and the like. The thin film may be of thickness between 5 and 500 nm. The thicknesses of each individual layer may be different or may be the same.

Dielectric layers: In some embodiments, nanostructured LSPR sensors will include one or more layers of a dielectric (insulating) material. In some embodiments, there may be about 1, 2, 5, 10, 15, 20, or more dielectric layers. Any of a variety of materials may be used, including, but not limited to, glass, ceramic, or polymer materials such as polyimides, heteroaromatic polymers, poly(aryl ether)s, fluoropolymers, or hydrocarbon polymers lacking polar groups. Polymer layers or thin films may be fabricated by any of a variety of techniques known to those of skill in the art, including, but not limited to, solution casting and spin coating, chemical vapor deposition, plasma enhanced chemical vapor deposition, and the like. In some embodiments, the surface plasmon resonance properties of a nanostructured LSPR sensor, e.g. resonance wavelength, may be tuned by adjusting the thickness or dielectric constant of the material used to form an insulating layer between two metallic layers.

Particles adsorbed to surfaces: In some embodiments, nanostructured or microstructured surfaces may be prepared by adsorbing or attaching particles, e.g. nanoparticles or fine particles, to substrate surface. The particles may be of any variety and of any shape including, but not limited to, spherical, non-spherical cubic, cuboid, pyramidal, cylindrical, conical, oblong, star-shaped, in the form of short nanowires, hollow, porous, and the like. Nanoparticles are particles of diameter ranging from 5 to 500 nanometers. Fine particles are particles of diameter ranging from 500 to 2,500 nanometers. Any of a number of different particle types may be used, including, but not limited to, metals, noble metals, metal-oxides, metal-alloys, metal-doped semi-conductors, non-metal composites, polymers, gold or silver nanoparticles, dielectric nanoparticles and microparticles, semiconductor nanoparticles, and hybrid structures such as core-shell nanoparticles, many of which are available commercially or can be prepared by any of a variety of methods known to those of skill in the art. Hybrid structures may be composed of different materials. For example, a core-shell nanoparticle may be comprised of a solid outer shell and a liquid inner core.

Coated particle surfaces: In some embodiments, nanostructured LSPR surfaces are prepared by adsorbing or attaching non-metallic nanoparticles to a substrate surface and coating or partially-coating the attached particles with a thin metallic film to create a capped-particle surface, e.g. a gold-capped particle surface. The nanoparticles may be coated with one or more layers of the thin metallic film. For example, the nanoparticles may be coated with about 1, 2, 5, 10, 20 or more layers of the thin metallic film. In some embodiments, the preferred metal for use in the thin metallic film will be noble metals such as gold, silver, platinum, palladium, copper, and the like. The thin metallic film may comprise any mixture and/or any combination of the preferred metals mentioned herein. For example, the thin metallic film may comprise of one layer of gold, one layer of copper, and one layer of a mixture of silver and platinum. The coating may be of thickness between 5 nm and 200 nm. In some embodiments, the nanostructured surface may cover the entire substrate surface. In other embodiments, the nanostructured surface may cover only a portion of the substrate surface, and may be distributed across the substrate surface in a predefined pattern.

Alternative nanostructured surfaces: In some embodiments, rather than utilizing nanoparticles adsorbed or attached to a surface to create nanostructured LSPR surfaces, the nanostructured surface may be fabricated using any of a variety of techniques known to those of skill in the art (e.g., patterned by mechanical, vacuum, or chemical methods). Nanostructures such as cylindrical columns or pillars, rectangular columns or pillars, cylindrical or rectangular nanowells, and the like may be fabricated in a variety of substrate materials using techniques such as photolithography and wet chemical etching, reactive ion etching, or deep reactive ion etching, focused ion beam milling, application of heat to metal thin films to form islands, dip-pen nanolithography, and the like.

Dimensions and patterns of nanostructures on surfaces: The dimensions of the aforementioned nanostructures may range from a few nanometers to hundreds of nanometers. In some embodiments, the nanostructured surface may cover the entire substrate surface. In other embodiments, the nanostructured surface may cover only a portion of the substrate surface, and may be distributed across the substrate surface in a predefined pattern. The sensor surface may be capable of sustaining a localized surface plasmon resonance over all or portion of the sensor surface. The nanostructured surface may be of high or low density. To measure properties of light transmitted through a sensor surface, having a nanostructured surface of low density may be desired. To measure properties of light reflected from a sensor surface, having a nanostructured surface of high density may be desired. A surface having a high density of nanostructures may absorb and scatter light efficiently. In some embodiments, it may be desirable to measure properties of light that is transmitted through the sensor surface. In some embodiments, it may be desirable to measure properties of light that is reflected from the sensor surface. For example, measuring properties of light reflected from the sensor surface may be superior than measuring light transmitted through the sensor surface in terms of plasmonic response to an analyte (see, e.g., O. Kedem et al., J. Phys. Chem. Lett., 2, 1223-1226, 2011).

Fabrication of the LSPR active surface: LSPR active surfaces may be created from the components described above in a variety of ways and/or steps. As a non-limiting, illustrative example, a method of creating one type of LSPR active surface mentioned herein may comprise 1) the deposition of a thin film of Au in the range of 5-500 nm thick, 2) chemistry deposition of nanometer size silica or polymer particles (˜10 to 2500 nm in size) in a random, close-packed configuration, and 3) capping of the silica or polymer particles with one or more layers of Au (˜5 to 200 nm thick).

Functional assays using Au core or Ag/Au core/shell nanoparticles and nanostructured LSPR surfaces: The nanoparticle-antibody conjugates or nanoparticle-antibody/enzyme conjugates (using either Au or Ag/Au nanoparticles, or hybrid magnetic/plasmonic nanoparticles as described elsewhere in this disclosure) are tested in functional assays against a metallic thin film LSPR surface modified with an antigen. If the nanoparticle-antibody conjugates (or nanoparticle-antibody/enzyme conjugates) are tested against an antigen not recognized by the antibody, the LSPR surface response is essentially flat. On the other hand, when the antibody does recognize the specific antigen, the resulting immobilization of the Au or Ag/Au nanoparticles (or hybrid magnetic/plasmonic nanoparticles) provides a large response from the LSPR sensor due to plasmon-plasmon coupling between the metal nanoparticles and the sensor surface. The ability to tune the plasmon resonance properties of both the nanoparticles (Au, Ag/Au, or hybrid magnetic/plasmonic nanoparticles) and the nanostructured LSPR surface to optimize plasmon-plasmon coupling-induced plasmon peak shift, and therefore assay sensitivity, is one of the unique features of the presently disclosed technology. Another beneficial property of using the metal (Au, Ag/Au, or hybrid magnetic/plasmonic) nanoparticles and nanostructured LSPR surfaces of the present disclosure is that the short-range distance-dependence for plasmon-plasmon coupling may be exploited to develop one-step homogeneous assays, i.e. assays where the initial molecular binding interaction takes place in solution, and that require no subsequent separation or rinse steps prior to detection. As a result of the enhanced sensitivity and simplified workflow for single-step, plasmon-plasmon coupling assays, such assays may also provide faster times-to-result (e.g. shorter assay readout times). In some embodiments, the assay time-to-result may be less than 5 minutes, less than 10 minutes, less than 15 minutes, less than 20 minutes, less than 25 minutes, less than 30 minutes, less than 40 minutes, less than 50 minutes, or less than 60 minutes. In some embodiments, the assay time-to-result may be more than 60 minutes, more than 50 minutes, more than 40 minutes, more than 30 minutes, more than 25 minutes, more than 20 minutes, more than 15 minutes, more than 10 minutes, or more than 5 minutes. In some embodiments, the assay time-to-result may be any value within this range, for example, about 18 minutes.

Types of plasmon-plasmon coupling assays: A variety of assays may be developed using the Au core nanoparticles, Ag/Au core/shell nanoparticles, or hybrid magnetic/plasmonic nanoparticles of the present disclosure (collectively referred to herein as “metal nanoparticles”) and any of a number of LSPR surfaces known to those of skill in the art. In preferred embodiments, the Au core and Ag/Au core/shell nanoparticles (or hybrid magnetic/plasmonic nanoparticles) of the present disclosure are combined with the use of nanostructured LSPR surfaces as described so that the plasmon resonance properties of the Au or Ag/Au nanoparticle and those of the nanostructured LSPR surface are substantially matched, thereby optimizing the observed plasmon peak shift and the detection sensitivity of the assay. Examples of assays that may be developed using these the disclosed compositions and methods include, but are not limited to, sandwich immunoassays (e.g. where the LSPR sensor surface is pre-functionalized with an affinity reagent that is specific for the analyte, and where an Au, Ag/Au or hybrid magnetic/plasmonic nanoparticle-conjugated detection antibody is used), competitive binding assays (e.g. where the LSPR sensor surface is pre-functionalized with an affinity reagent that is specific for the analyte, and the presence of the analyte in a sample is detected by incubating the sensor surface with a mixture of the sample and a solution comprising a metal nanoparticle (Au, Ag/Au, or hybrid magnetic/plasmonic) conjugated to a known ligand for the affinity reagent; in an alternate implementation of a competitive assay, the surface is functionalized with the antigen to be detected and the sample is pre-incubated with a solution containing metal nanoparticles (Au, Ag/Au, or hybrid magnetic/plasmonic) conjugated to a ligand capable of recognizing the antigen), hybridization assays (e.g. where the LSPR sensor surface is pre-functionalized with an oligonucleotide capture probe that is capable of specific hybridization to part of a target oligonucleotide, and where an Au, Ag/Au, or hybrid magnetic/plamonic nanoparticle-conjugated oligonucleotide detection probe that is capable of specific hybridization to part of the target oligonucleotide is also used), and the like. The assays may be qualitative or quantitative, and in some embodiments may also be multiplexed, that is, capable of simultaneous detection of more than one analyte. The assay readout may be qualitative, e.g. through visual observation of a color change in light reflected from the sensor surface, or may be quantified through the use of an optical reader to measure precise shifts in plasmon resonance peak or other physical properties (e.g. intensity, polarization, angle of reflection, RGB or greyscale values, etc.) of light reflected or transmitted by the sensor surface.

Analytes: The compositions, methods, devices, and systems of the present disclosure may be used for detection and/or quantitation of analytes (markers, biomarkers) present in small, moderate, or large quantities in a sample. The analyte may be any molecule of interest. The analyte may be a peptide, a protein, an oligonucleotide, a DNA molecule, an RNA molecule, a virus, a bacterium, a cell, a pathogen, a lipid molecule, a carbohydrate molecule, a small organic molecule, a drug molecule, or an ion. The analyte may be a biomarker of interest in clinical diagnostic applications, e.g. creatinine, lactate, C-reactive protein, alpha-fetoprotein, or cardiac marker tests (e.g. cardiac troponin I (cTnI), cardiac troponin T (cTNT), cardiac phosphocreatine kinase M and B (CK-MB), and brain natriuretic peptide (BNP)), cortisol, S100BB, tau protein, thyroid-stimulating hormone (TSH) or circulating tumor cells (CTC's).

Samples: Assays for the detection and quantitation of analytes in a variety of samples may be implemented using the Ag/Au nanoparticles and nanostructured LSPR sensors of the present disclosure. Examples of samples include air, gas, water, soil, or industrial process stream samples, as well as biological samples such as feces, tissue, cells, or any bodily fluid, such as blood, plasma, serum, sweat, tears, urine, cerebral spinal fluid, or saliva. Biological samples may comprise or be derived from virus, bacteria, pathogens, plants, animals, or humans. In some embodiments, samples derived from animals or humans may be “patient samples”, and the results of the assay may be used in pathogen detection, disease diagnosis, or the making of treatment and healthcare decisions by a healthcare provider.

Affinity reagents: In some embodiments, one or more primary binding components (affinity reagents, affinity tags) may be pre-immobilized on the sensor surface prior to performing an assay using any of a variety of attachment chemistries known to those of skill in the art. In some embodiments, one or more primary binding components may be mixed with the sample prior to contacting the sensor surface with the sample (e.g., as part of the assay procedure). In some embodiments, one or more secondary binding components may also be used to confer high specificity and enhanced sensitivity to the performance of the LSPR-based assay. In many embodiments, the secondary binding component may be conjugated to a sensitivity enhancing label such as the Au or Ag/Au nanoparticles described above to further increase the sensitivity of the assay. Examples of suitable primary and secondary binding components for use in the methods and devices disclosed herein include, but are not limited to, antibodies, antibody fragments, aptamers, molecularly imprinted polymers, biotin, streptavidin, his-tags, chelated metal ions such as Ni-NTA, receptors, enzymes, peptides, proteins, and oligonucleotide probes.

Optical readers: In some embodiments, the Au and/or Ag/Au nanoparticles and/or LSPR surfaces disclosed herein are used in conjunction with optical devices and instruments (e.g. optical readers) for quantifying the plasmon peak shifts observed in assays performed using Au or Ag/Au nanoparticle-conjugated affinity reagents, thereby improving both assay quantitation and assay sensitivity. In some embodiments, optical instruments may be designed to illuminate the LSPR sensor surfaces from the back side, in which case it is desirable for the substrate material to be optically transparent. In other embodiments, the sensor surface may be illuminated from the front side, and the transparency or opacity of the sensor substrate material is not important. In some embodiments, it may be desirable to measure properties of light that is transmitted through the sensor surface. In many embodiments, it is desirable to measure properties of light that is reflected from the sensor surface. For example, measuring the properties of light reflected from the sensor surface may be superior to measuring light transmitted through the sensor surface in terms of the ability to monitor the plasmonic response to an analyte. Any of a variety of physical properties of the light transmitted by or reflected from the LSPR sensor surface may be measured, e.g. spectra and/or spectral shifts, intensity, polarization, angle of reflection, or change in RGB or greyscale values. Optical devices and instruments suitable for use with the plasmonic nanoparticles and LSPR sensor surfaces described herein will typically include one or more light sources, detectors, and other optical components, e.g. lenses, mirrors, filters, beam-splitters, prisms, polarizers, optical fibers, as well as microprocessors, computers, computer readable media, and the like.

Light sources: The light source may be sun light, room light, an LED, laser, halogen source, or any other suitable light source. The light source may direct light at the sensor surface before, during, and/or after an assay reaction takes place on the sensor surface. In some embodiments, the light source will be shuttered so that the sensor surface may be illuminated at selected times. In some embodiments, the light source may be pulsed at a pre-specified frequency so that signal-to-noise ratios for detection of the transmitted or reflected light may be improved through frequency-dependent amplification or boxcar integration techniques. The light source may direct light to the LSPR sensor surface from the substrate side or from the sensor surface side. The light source may be placed such that light is generally incident on the LSPR surface at an angle of 90 degrees to the LSPR sensor surface (perpendicular illumination). Similarly, a detector may be placed such that it detects light that is reflected from the surface at 90 degrees. Alternatively, the light source may be placed such that light is generally incident on the LSPR surface at an oblique angle. Similarly, the detector may be placed such that it detects the reflected light from the surface at an oblique angle. The light source may be directed through an optical waveguide or an optical fiber. The optical channel or optical fiber may then be positioned so that light exits the optical waveguide or optical fiber and is incident on the LSPR surface at the desired angle. In some embodiment, the light source illumination may be directed through a set of lenses, mirrors, and/or beamsplitters to impinge on the surface at the desired angle. In some embodiments, the light source may provide broad band (e.g. white) light. In other embodiments, the light source may be configured to provide narrow-band light. Often, the light source and illumination system will be configured to provide collimated light.

Detectors: The detector may be a photodiode, avalanche photodiode, photomultiplier tube, an image sensor, or any other form of suitable light detector. In some embodiments, one or more detectors may be used to detect light transmitted by or reflected light from the LSPR sensor surface before, during, and/or after the assay is performed, thereby enabling the collection of endpoint assay determinations and/or kinetic assay data. As indicated above, in some embodiments, an image sensor may be used. Examples of suitable image sensors include CCD sensors, CMOS sensors, or NMOS sensors. The image sensor may capture a series of one or more images of all or part of the LSPR sensor surface. In some embodiments, the image sensor may capture images of more than one LSPR sensor surfaces. The series of images may be greyscale images or RGB images. The series of images may include images captured before, during, and after an assay is completed. In some embodiments, the series of images may be of sufficient spatial resolution that a localized change in plasmon resonance peak due to the presence of an analyte may be detected over the course of a series of time lapse images. The series of images may comprise about or more than 1000 images, 500 images, 400 images, 300 images, 200 images, 100 images, 50 images, 10 images, 5 images, 4 images, 3 images, or 2 images. The image sensor may capture the series of image frames at a predefined capture rate. The inverse of the capture rate may be 1 millisecond per frame, 2 milliseconds per frame, 5 milliseconds per frame, 10 milliseconds per frame, 20 milliseconds per frame, 50 milliseconds per frame, or any capture rate that provides acceptable signal-to-noise ratios under the set of illumination conditions employed. Image sensors may vary in terms of pixel size and pixel count. The image resolution may depend on the pixel size and pixel count. Image sensors may have a pixel count of about or more than 0.5 mega pixels, 1 mega pixels, 4 mega pixels, 10 mega pixels, 20 mega pixels, 50 mega pixels, 80 mega pixels, 100 mega pixels, 200 mega pixels, 500 mega pixels, or 1000 mega pixels. The pixel size corresponding to the image sensor may be about or less than 5 microns, 3.5 microns, 2 microns, 1 micron, 0.5 microns, or 0.1 micron.

Illumination and collection optics: As indicated above, optical devices and instruments suitable for use with the plasmonic nanoparticles and LSPR sensor surfaces described herein will typically also include other optical components, e.g. lenses, mirrors, filters, beam-splitters, prisms, polarizers, optical fibers, and the like, for assembly of illumination and collection optical sub-systems. In some embodiments, an epi-illumination design may be used such that a single objective lens (or an equivalent optical setup using multiple lenses) acts to both deliver illumination light to the LSPR sensor surface and collect reflected light from the LSPR sensor surface. The objective lens (or an equivalent optical setup using multiple lenses) may provide a magnification of the sensor surface. In some embodiments, the objective (or an equivalent optical setup using multiple lenses) may have long working distance (e.g., 2-5 mm) to provide enough clearance to accommodate fluidic systems designed to deliver samples and assay reagents to the sensor surface. In some embodiments, the objective lens may be optimized for near-field imaging. The optical system may provide an overall magnification that is about 0.5×, 1×, 5×, 10×, 20×, 50×, 100×, 200×, or higher. The magnification of the optical system enables each pixel of the image frame to correspond to a surface area that is much smaller than the pixel size. For examples, an image sensor with a pixel size of 5 microns capturing an image using a 10x objective will produce an image with a pixel that corresponds to a sensor surface of 0.25 um². This magnification may enable local areas on the LSPR surface corresponding to plasmon-plasmon coupling activity resulting from presence of the analyte to be clearly distinguishable and counted. In some embodiments, the optical illumination and collection paths are designed to work with the LED and the camera of a smartphone.

Data reduction and analysis: The signals or images acquired by the one or more detectors of the optical system may be analyzed using algorithms to improve signal-to-noise ratios and assay sensitivity. Algorithms may be stored in a computer readable medium. The computer readable medium may be any medium capable of storing data in a format that may be read or processed by a device (e.g., compact disc, floppy disk, USB flash drive, hard disk drive, etc). Examples of algorithms that may be usefully employed include, but are not limited to, signal averaging algorithms, signal smoothing algorithms (e.g. the Savitsky-Golay algorithm), signal histogramming and determination of the moments of the histogram distribution, pattern mining algorithms that delineate areas of the sensor surface that exhibit response to contact by an analyte, and the like. The pattern mining algorithms may manipulate changes in RGB or greyscale values to determine specific patterns on an image (e.g., determining areas of an LSPR sensor surface for which image pixels have undergone a change in red pixel value within a certain defined range). In some embodiments, the algorithm may determine a concentration of the analyte in a sample. Several known concentrations of the analyte and a corresponding signal that they generate may be measured and used for the generation of a calibration curve. An analyte may be detected as described herein, and the signal measured may then be compared to the calibration curve to determine a concentration of the analyte in a sample.

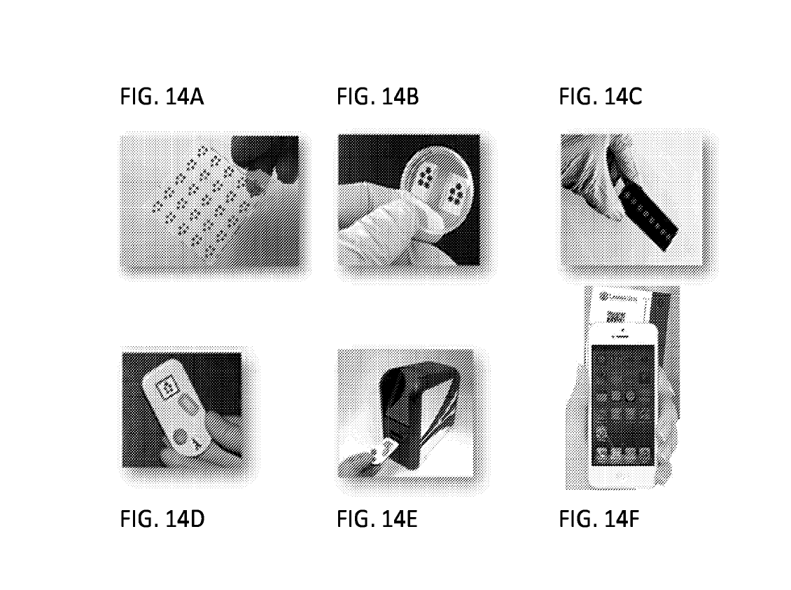

Microfluidic devices and systems: The compositions, methods, devices, and systems of the present disclosure may utilize a fluidic system (e.g. a microfluidic device or fluidic device) that is fully or partially integrated with one or more LSPR sensors (FIGS. 14A-F). Often, the fluidic system will be configured to deliver one or more samples and/or assay reagents to the sensor surface. Typically, the fluidic system will contain one or more pumps (or other means of fluid actuation), valves, fluid channels or conduits, membranes, flow cells, reaction wells or chambers, and/or reagent reservoirs with reagents necessary for carrying out the assay. In some embodiments, all or a portion of the fluidic system components may be integrated with the LSPR sensor to create LSPR chips or devices. In some embodiments, the LSPR chips or devices may be disposable or consumable devices. In some embodiments, all or a portion of the fluidic system components may reside in an external housing or instrument with which the LSPR sensor chip or device interfaces.

Fluid actuation mechanisms: In some embodiments, the fluidic system may include one or more fluid actuation mechanisms. Examples of suitable fluid actuation mechanisms for use in the disclosed methods, devices, and systems include application of positive or negative pressure to one or more reaction wells or reagent reservoirs, electrokinetic forces, electrowetting forces, passive capillary action, capillary action facilitated through the use of membranes and/or wicking pads, and the like. Positive or negative pressure may be applied directly, e.g. through the use of mechanical actuators or pistons that are coupled to the reservoirs to actuate flow of the reagents from the reservoirs, through the fluidic channels or conduits, and onto the sensor surface. In some embodiments, the mechanical actuators or pistons may exert force on a flexible membrane that is used to seal the reservoirs. In some embodiments, positive or negative pressure may be applied indirectly, e.g. through the use of a pressurized gas lines or vacuum lines connected with one or more reservoirs. In some embodiment, pumps may be used to drive fluid flow. These may be pumps located in a housing or instrument with which an LSPR sensor interfaces, or in some embodiments they may be microfabricated pumps integrated with the sensor. In some embodiments, fluid flow may be driven by centrifugal forces, e.g. by using a spinning or rotating mechanism, device, or system.

Fluid channels: In some embodiments, the fluid channels or conduits may have a substantially rectangular cross-section. In these embodiments, the fluid conduits may have a width of about 10 um to about 5 mm, and a depth of about 10 um to 5 mm. In other embodiments, the fluid conduits may have a substantially circular cross-section. In these embodiments, the fluid conduits may have a diameter of between about 10 um and 5 mm.

Valves: In some embodiments, the fluidic system may include one or more valves for switching fluid flow between reservoirs and channels. These may be valves located in a housing or instrument with which an LSPR sensor chip interfaces, or in some embodiments they may be microfabricated valves integrated with the sensor chip. Examples of suitable valves for use in the disclosed devices and instruments include solenoid valves, pneumatic valves, pinch valves, membrane valves, and the like.

Reaction wells: The LSPR sensor chips disclosed herein may have one or more reaction wells containing an LSPR sensor where an assay takes place. Some of the reaction wells may be control wells. The combination of fluid actuation mechanisms and control components, e.g. pumps and valves, used in the fluidic system allows different samples and reagents from the reservoirs to be mixed and introduced into the reaction wells as required to perform a specific assay. For example, the LSPR sensor chip may contain a sample reservoir. In some embodiments, the sample to be assayed may be deposited into the sample reservoir, and the sample may then be introduced from the sample reservoir into one or more reaction wells using pumps, valves, and fluid conduits. The reaction wells may be aligned with the LSPR sensor surface(s), which may react with the sample to produce a shift in the plasmon resonance peak of light reflected from the sensor surface(s). In some embodiments, the sample to be assayed may be deposited onto the LSPR sensor surface by depositing the sample directly into the reaction well. In some embodiments, single step assays are performed by mixing the sample with a secondary binding component, e.g. an Au or Ag/Au nanoparticle-conjugated secondary binding component, either before pipetting into the LSPR sensor device, or within a reaction well of the LSPR sensor device, and the presence of the analyte is detected directly without the need for separation or rinse steps. The diameter of the reaction wells may range from 500 μm (or smaller) to 5 mm in diameter. The reaction wells need not be circular in shape. In some embodiments, the cross-sectional area of the reaction wells may range from about 25 μm² to about 25 mm². In some embodiments, the depth of the reaction wells may range from about 10 μm to about 10 mm deep. For example, the depth of the reaction well may be around 35 μm. In some embodiments, the volume of the reaction wells may range from 100 nanoliters to 3 milliliters. In some embodiments, the reaction wells may be configured to hold a volume of less than 25 μL. In some embodiments, the LSPR sensor chip may have a plurality of reaction wells, wherein each reaction well contains a sensor. In some embodiments, the LSPR sensor chips may have a single reaction well containing an array of sensors. The LSPR sensors may be multi-paneled or multiplexed, such that a different type of assay may be run in each reaction well. Thus, different reaction wells may contain different types of sensors, including unmodified sensors and sensors with primary binding components (affinity reagents) immobilized thereon. In some embodiments, some of the reaction wells may be control wells.

Reservoirs: In some embodiments, the LSPR sensor chip may include one or more sample or reagent reservoirs. In some embodiments, the sample to be assayed may be deposited onto the LSPR sensor surface by depositing the sample directly into the sample reservoir. In some embodiments, the sample or reagents in the reservoirs may be introduced onto the sensor surface through the fluid channels, by using pumps, valves, and/or membranes. In general, the reservoirs may contain samples, reagents, diluents, conjugated antibodies, particles or beads, and/or waste products resulting from running an assay. In some embodiments, the LSPR sensor device may contain reservoirs which contain pre-loaded assay reagent(s). When the sample is introduced into these reservoirs, the sample is mixed with the reagent(s) and the mixture may then flow into the reaction wells where the assay takes place. Further, the LSPR sensor chip may also contain one or more waste reservoirs. In some embodiments, the reservoirs may have a diameter of about 2 mm to about 10 mm, and a depth of about 0.1 mm to about 5 mm, or may have dimensions such that the volume is between 1 nL and 3 mL.

Lyophilized or dry colloid conjugates: Single step assays require the sample to be pre-mixed with a secondary binding component, e.g. an Au or Ag/Au nanoparticle-conjugated secondary binding component, before the mixture reaches the LSPR sensor device. In one embodiments, the Au or Ag/Au nanoparticle-conjugated secondary binding component can be lyophilized in a small bead and placed in the channel upstream from the LSPR sensor. In another embodiments, the Au or Ag/Au nanoparticle-conjugated secondary binding component can be dried in the channel upstream from the LSPR sensor. In both previous embodiments, the test sample will rehydrate the lyophilized bead or dried Ag/Au conjugate, mix with the conjugates, and ferry them towards the sensing surface.

Membranes: In some embodiments, there may be one or more membranes that serve as a filter placed on top of sample reservoirs and/or upstream of reaction wells. In some embodiments, the sample to be assayed may be deposited onto the LSPR sensor surface by depositing the sample directly onto a membrane filter that covers the reaction well. The membrane filter may be designed to filter out unwanted particles according to size. For example, the filter may contain appropriately sized pores that only allow smaller sized particles to filter through to the reaction wells. Unwanted particles may include cells, salts crystals, insoluble precipitates, or other particulates which may interfere with the assay or clog fluid channels. A sample may contain one or more molecules of interest which may be separated by the membrane. Thus, different types of molecules may filter through to different reaction wells, and membranes of different porosity or different selectivity may enable the concurrent analysis of more than one analyte in a sample. In some embodiments, the sample is introduced by depositing it over a reservoir instead of or in addition to depositing it into a reaction well. The LSPR sensor may contain one or more reservoirs especially adapted to receive samples. The sample reservoirs may or may not include membranes placed on top of the reservoirs depending on whether or not filtering is desired. Filtration may be achieved by mechanically applying pressure on the sample with, for example, using a piston. When the piston applies pressure on the sample, the smaller particles may be forced through the filtration membrane while the larger particles do not pass through the filtration membrane. Filtration may also be achieved without applying positive mechanical pressure. For example, filtration may be achieved by gravitational forces or through negative pressure applied from the side of the filtration membrane opposite where the sample lies. Alternatively, filtration may be achieved by capillary draw through membranes and/or wicking pads.

Fabrication materials, techniques, and dimensions: In general, the reaction wells, sample and reagent reservoirs, and fluid channels may be fabricated using any of a variety of materials, including, but not limited to glass, fused-silica, silicon, polycarbonate, polymethylmethacrylate, cyclic olefin copolymer (COC) or cyclic olefin polymer (COP), polydimethylsiloxane (PDMS), or other elastomeric materials. Suitable fabrication techniques (depending on the choice of material) include, but are not limited to CNC machining, photolithography and etching, laser photoablation, injection molding, hot embossing, die cutting, and the like.

The size and shape of the fluidic channels, as well as the pressure applied to the one or more reaction wells or reservoirs, may be designed such that flow into the reaction wells is laminar. In some embodiments, the length of the fluid conduits may range from about 1 mm to about 100 mm. In some embodiments, the fluid conduits may be have a substantially rectangular cross-section. In these embodiments, the fluid conduits may have a width of about 10 um to about 5 mm, and a depth of about 0.1 mm to 2.5 mm. In other embodiments, the fluid conduits may have a substantially circular cross-section. In these embodiments, the fluid conduits may have a diameter of between about 10 um and 5 mm.

Kits: Also disclosed herein are kits that comprise the nanoparticle compositions, conjugated assay reagents, LSPR sensors, and LSPR sensor devices described above. In some embodiments, kits may comprise the Ag/Au core/shell nanoparticles described above. In some embodiments, the kits may comprise Ag/Au core/shell nanoparticles and reagents for use in performing bioconjugation reactions with user-supplied antibodies, antibody fragments, proteins, or other binding components. In some embodiments, the kits may comprise one or more Ag/Au core/shell nanoparticle-conjugated detection antibodies or other conjugated binding components. In some embodiments, the kits may further comprise the nanoparticle-conjugated detection antibodies or other conjugated binding components and LSPR sensors having surfaces that have been pre-functionalized with appropriate capture antibodies. In some embodiments, the kits comprising LSPR sensors may further comprise coupling reagents for functionalizing the LSPR sensor surfaces with a capture antibody or other binding component of the user's choice. In some embodiments, one or more LSPR sensors may be packaged in one or more test strips or microfluidic devices as described above. In any of these embodiments, the kits may further comprise other assay reagents, e.g. buffers, salt solutions, enzymes, enzyme co-factors, enzyme inhibitors, enzyme substrates, antibodies or antibody fragments, proteins, peptides, oligonucleotides, and the like.

Single-step point-of-care (POC) diagnostic assays: One of the most sought after and desired capabilities of modern diagnostics is a single step, surface-based homogeneous assay that is precisely quantitative, requires no wash step(s), and reaches into the low to sub-picogram levels of detection. We have developed such assay techniques using the Au or Ag/Au nanoparticles and nanostructured LSPR sensor surfaces described above, for example, sandwich immunoassay formats have been developed where the presence of an analyte in the sample results in formation of a bound complex between a primary binding component immobilized on the nanostructured LSPR surface, the analyte, and a secondary binding component conjugated to an Au or Ag/Au nanoparticle. Single-step assays exhibiting limits of detection (LODs) in the low picogram/mL range have been demonstrated for a number of relevant clinical diagnostic model systems, two of which are highlighted below. Optimization of assay parameters, e.g. optimization of the choice and density of immobilized primary binding components on the sensor surface, assay buffers, assay incubation times, etc., and of detection parameters, e.g. the intensity and/or wavelength of light used to illuminate the sensor surface, the choice of low noise detector, etc., may push the achievable detection limits much lower than those demonstrated in the following examples. In some embodiment, the limit of detection may be less than 1 mg/ml, less than 100 ug/ml, less than 10 ug/ml, less than 1 ug/ml, less than 100 ng/ml, less than 10 ng/ml, less than 1 ng/ml, less than 100 fg/ml, less than 10 fg/ml, less than 1 fg/ml, or less than 0.1 fg/ml.

### Example 1—Alpha-Fetoprotein (AFP) Detection Using an LSPR Single-Step (Homogeneous) Plasmonic Assay

Alpha-fetoprotein (AFP) is commonly known for its use in prenatal screening for risk assessment of fetal distress situations and genetic disorders. Also of importance, the function of AFP in adult humans has been linked to several pathologies. For instance, in men, non-pregnant women, and children, elevated AFP levels in the blood can indicate the presence of certain types of cancers, such as cancer of the testicles, ovaries, stomach, pancreas or liver. High levels of AFP may also be found in lymphoma, Hodgkin's lymphoma, brain tumors and renal cell cancer.

Given the broad dynamic range of AFP levels found in patients, the disclosed LSPR biosensor platform technologies have been used to develop several robust assay formats to precisely quantitate AFP found in human plasma, serum specimens and whole blood. AFP at pre-natal levels in humans (>100-1000 ng/mL) can be precisely measured and quantitated in less than 10 minutes using the disclosed LSPR biosensor in a single-step format. Additionally, adult human AFP levels of ˜1 ng/mL to 300 ng/mL can be precisely quantitated in 15 minutes. AFP levels of <7 ng/mL fall below the lower limit of quantitation (LLOQ) for most currently available central laboratory commercial AFP tests—all of which require multiple wash steps and as many as 4 hours of assay time to complete.

The single-step AFP assay disclosed herein is a one-step, 15 minute assay. The assay requires minimal intervention by an end user or practitioner and is adaptable to a number of existing industry products. An Au-conjugated-anti-AFP detection antibody solution is mixed with the sample immediately prior to injection of the mixture onto an LSPR diagnostic sensor pre-functionalized with an anti-AFP capture antibody. The assay readout (e.g. measurement of the shift in absorption peak for light reflected from the LSPR surface) in this example occurs 15 minutes after the injection. In some cases, the assay readout time (or time-to-result) may be either longer or shorter than 15 minutes. Assay readout times and times required to report test results may range from about 10 minutes to about 20 minutes. These times compare favorably with those required for traditional ELISA-based AFP assays, which typically take from 90 to 120 minutes to perform. The single-step plasmonic assay does not require wash steps and can be automated for quantitative, facile, and medium or high-throughput analysis. The data shown in FIG. 4A demonstrate that the LSPR biosensor response induced by the presence of AFP is linear with concentration over the range of 1.2 ng/mL to >100 ng/mL. Assay data collected using five independent biosensors over this AFP concentration range had a 9.0%CV. For assay data collected for several independent runs, the inter-assay reproducibility had % CVs of well below 20% across the concentration range of 1.23 ng/mL-300 ng/mL, with an R² value of 0.984. The single-step AFP assay demonstrates an LOD of 1.2 ng/mL, well below the value of 5-10 ng/mL reported in the package inserts for several commercial (central laboratory) ELISA-based AFP kits.

### Example 2—Salivary Cortisol Detection Using an LSPR Competitive Single-Step Plasmonic Assay

Stress is a leading cause of morbidity and mortality in the United States. It also represents a significant expense for businesses due to the ballooning costs of employer's sponsored healthcare, and loss of employee productivity due to sick leave or time off

The emerging medical consensus is that cortisol is a good biomarker for stress, because it is linked with many physiologic processes. Besides stress, cortisol is also an indicator of several diseases. For instance, increased cortisol production is associated with Cushing syndrome, while decrease of cortisol production is associated with adrenal insufficiency (Addison's disease).

Cortisol is the end product of the hypothalamic pituitary-adrenal (HPA) axis. In a healthy human, cortisol production follows a circadian rhythm. Cortisol levels peak in the early morning and drop to the lowest concentration at night. The normal level of cortisol in blood depends on the age and gender of the individual. As a general guideline though, cortisol levels in adults are ˜50-230 ng/mL in the early morning, and ˜30-160 ng/mL in the afternoon. In response to stress, cortisol levels rise independently of the circadian cycle for all groups of individuals. After appraisal of the stressor, the hypothalamus triggers a signaling cascade that culminates with the release of cortisol into the blood stream. Blood cortisol concentrations peak about fifteen minutes after the onset of a stressor.

Measuring biomarkers in a blood-based assay format requires either a blood draw performed by trained personnel in a medical setting or a finger prick. A saliva-based cortisol assay would palliate shortcomings of a blood-based assay. For instance, monitoring cortisol in saliva opens a window of opportunity to conduct convenient stress-related research and testing that involves many repeated measures from a broad pool of persons in both clinical and non-clinical settings.

Salivary measurements of small steroids such as cortisol take advantage of the fact that free cortisol is lipid soluble; this biologically active fraction of total cortisol passes through the acinar cells to enter saliva via passive diffusion in proportion to cortisol levels in blood. A major drawback though is that the level of cortisol in saliva is a fraction of the levels in blood (5%-10%). For instance, salivary cortisol in adults is in the range of 0.3-15 ng/mL in the mornings and drops to 0 (non-detectable)-3.6 ng/mL at night. Hence, high-sensitivity is required for precise saliva based cortisol testing.

We describe here a high-sensitivity, quantitative, single-step, 15 min assay for precise cortisol monitoring using a LSPR plasmonic platform. The assay is based on competition between cortisol in saliva and cortisol-labeled colloidal gold in the assay diluent buffer. With low levels of endogenous cortisol, the cortisol-labeled moiety is captured on the LSPR plasmonic surface where it generates a measurable color change of the surface. In contrast, binding sites of the LSPR surface are saturated at high levels of endogenous cortisol, leaving no room to bind for the cortisol-labeled moiety. Hence, no color change of the LSPR surface happens. For intermediate endogenous cortisol levels, competition takes place between free cortisol and the cortisol-labeled moiety. This results in a gradual change in the LSPR plasmonic surface color that can be precisely quantified using a spectrometer or a digital camera.

The LSPR plasmonic sensors disclosed herein have been used to precisely quantitate cortisol over the range of 50-10,000 pg/mL in just 15 minutes. FIG. 4B shows data for LSPR biosensor response induced by the presence of cortisol in the sample. The assay utilizes an Au-conjugated-anti-cortisol detection antibody, and cortisol pre-functionalized LSPR surface. For data collected using several different biosensors, the coefficient of variation (%CV) had values of ˜20% at cortisol concentrations of 130 pg/mL and less than 8% for concentrations of 390-1000 pg/mL. When a known amount of cortisol is added in the sample, the recovery values of 93-105% of the nominal spiked value were measured across the entire 130-1000 pg/mL range.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A nanoparticle composition comprising:
a) a silver (Ag) core;
b) a gold (Au) shell partially or wholly encapsulating the silver core, wherein the thickness of the gold shell is substantially less than the diameter of the silver core; and
c) a polymer layer partially or wholly encapsulating the Ag core and the Au shell.

2.-4. (canceled)

5. The nanoparticle composition of claim 1, wherein the gold shell has a thickness of between 1 and 20 atomic layers.

6. (canceled)

7. The nanoparticle composition of claim 1, wherein the polymer layer is between 1 nm and 50 nm thick.

8.-11. (canceled)

12. The nanoparticle composition of claim 1, wherein the nanoparticle has an average dimension ranging from 20 nm to 80 nm.

13. (canceled)

14. The nanoparticle composition of claim 1, further comprising a biomolecule layer conjugated to the gold shell.

15. (canceled)

16. The nanoparticle composition of claim 14, wherein the biomolecule layer is conjugated to the thin gold shell using a bifunctional cross-linker comprising a mercapto group.

17. A method for producing core-shell nanoparticles comprising:
a) reducing silver ions in solution to metallic silver, thereby producing silver (Ag) core nanoparticles;
b) rinsing the silver colloidal particles produced in step (a) to produce silver core nanoparticles having a stable plasmon resonance peak in the range of 400-680 nm; and
c) growing an epitaxial gold (Au) shell on the silver core nanoparticles produced in step (b) in the presence of a polymer solution to thereby generate Ag/Au core-shell nanoparticles.

18.-21. (canceled)

22. The method of claim 17, wherein the polymer has a molecular weight in the range of 3,500 Da to 50,000 Da.

23. The method of claim 17, wherein a ratio of a concentration of the polymer to a concentration of the silver core nanoparticles used in step (c) has a value in the range of 10³ to 10⁹.

24.-39. (canceled)

40. A method for detection of analytes in a sample comprising:
a) mixing a sample containing one or more analytes of interest with one or more secondary binding components conjugated to metal nanoparticles, wherein the one or more secondary binding components are capable of specifically binding to the one or more analytes of interest;
b) contacting an LSPR surface with the mixture of step (a), wherein the LSPR surface has been functionalized with one or more primary binding components that are capable of specifically binding to the one or more analytes of interest; and
c) detecting a change in a physical property of light transmitted by or reflected from the LSPR surface;
wherein the plasmon resonance properties of the metal nanoparticles and those of the LSPR surface are adjusted to substantially match.

41. The method of claim 40, wherein the metal nanoparticles are selected from the group consisting of Au nanoparticles, Ag/Au core/shell nanoparticles.

42.-44. (canceled)

45. The method of claim 41, wherein the plasmon resonance properties of the Ag/Au core/shell nanoparticles are adjusted by a method selected from the group consisting of changing the size of Ag core nanoparticles used to fabricate the Ag/Au core/shell nanoparticles, changing the shape of Ag core nanoparticles used to fabricate the Ag/Au core/shell nanoparticles, changing the thickness of an Au shell used to fabricate the Ag/Au core/shell nanoparticles, and any combination thereof.

46. The method of claim 40, wherein the LSPR surface is a nanostructured LSPR surface.

47. The method of claim 46, wherein the plasmon resonance properties of the nanostructured LSPR surface are adjusted by a method selected from the group consisting of changing the choice of materials used to fabricate the LSPR surface, changing the dimensions of the layers of material used to fabricate the LSPR surface, changing the number of layers of material used to fabricate the LSPR surface, changing the order of the layers used to fabricate the LSPR surface, and any combination thereof.

48.-51. (canceled)

52. The method of claim 40, wherein a limit of detection (LOD) for the method is better than 1 ug/mL.

53. (canceled)

54. The method of claim 40, wherein a limit of detection (LOD) for the method is better than 100 pg/mL.

55.-58. (canceled)

59. The method of claim 40, wherein the method is performed as a single-step assay that provides a result in 30 minutes or less.

60. (canceled)

61. A system for detection of one or more analytes in a sample comprising:
a) one or more detection probes capable of specific binding or hybridization with the one or more analytes, wherein the one or more detection probes are conjugated to metal nanoparticles; and
b) one or more nanostructured LSPR surfaces, wherein the one or more nanostructured LSPR surfaces are pre-functionalized with one or more primary binding components capable of specific binding or hybridization with the one or more analytes;
wherein the plasmon resonance properties of the metal nanoparticles and those of the one or more nanostructured LSPR surface have been adjusted to substantially match in order to optimize detection sensitivity; and
wherein the formation of bound complexes between the one or more detection probes, the one or more analytes, and the one or more primary binding components on the one or more nanostructured LSPR surfaces produces a detectable change in a physical property of light transmitted by or reflected from the one or more nanostructured LSPR surfaces.

62. The system of claim 61, wherein the metal nanoparticles are selected from the group consisting of Au nanoparticles, Ag/Au core/shell nanoparticles.

63. (canceled)

64. The system of claim 62, wherein the plasmon resonance properties of the one or more nanostructured LSPR surface have been adjusted by a method selected from the group consisting of changing the choice of materials used to fabricate the LSPR surface, changing the dimensions of the layers of material used to fabricate the LSPR surface, changing the number of layers of material used to fabricate the LSPR surface, changing the order of the layers used to fabricate the LSPR surface, and any combination thereof.

65.-68. (canceled)

69. The system of claim 62, wherein the physical property of light is selected from the group consisting of intensity, spectrum, polarization, angle of reflection, and changes in RGB or greyscale value.

70. The system of claim 62, wherein a limit of detection (LOD) for the method is better than 1 ug/mL.

71. (canceled)

72. The system of claim 62, wherein a limit of detection (LOD) for the method is better than 100 pg/mL.

73.-78. (canceled)

79. The system of claim 62, wherein the one or more pre-functionalized, nanostructured LSPR surfaces are packaged within a disposable fluidic device that further comprises fluidic components selected from the group including fluid channels, reaction wells, sample reservoirs, reagent reservoirs, and any combination thereof.

80. The system of claim 79, wherein the disposable fluidic device interfaces with an instrument that comprises additional components selected from the group consisting of light sources, detectors, cameras, lenses, mirrors, filters, beam-splitters, prisms, polarizers, optical fibers, pumps, valves, microprocessors, computers, computer readable media, and any combination thereof.

81.-109. (canceled)
